(19) United States
(12) Patent Application Publication
(10) Pub. No.: US 2025/0344942 A1
(43) Pub. Date: Nov. 13, 2025

(54) MULTISPECTRAL IMAGING CAMERA AND METHODS OF USE

(71) Applicant: Vicarious Surgical Inc., Waltham, MA (US)

(72) Inventors: Justin Keenan, Lexington, MA (US); Marshall Wentworth, Waltham, MA (US); Michael Cafferty, Waltham, MA (US); Zach Sherin, Cambridge, MA (US); Ashish Panse, Burlington, MA (US)

(21) Appl. No.: 18/868,449

(22) PCT Filed: May 25, 2023
(86) PCT No.: PCT/US2023/023593

§ 371 Date: Nov. 22, 2024

Related U.S. Application Data
(60) Provisional application No. 63/345,800, filed on May 25, 2022.

(51) Int. Cl. A61B 1/04; A61B 1/00; A61B 1/06; A61B 90/00

(52) U.S. Cl. CPC A61B 1/043; A61B 1/00197; A61B 1/0638; A61B 1/0646; A61B 1/0684; A61B 90/37; A61B 2090/371; A61B 2090/373

(57) ABSTRACT

A surgical robotic system and method of providing simultaneous multispectral imaging are disclosed herein. In some embodiments, the system includes a first and second camera assembly having one or more LEDs, one or more lens, one or more filter elements and one or more imaging sensors, the first and second camera assembly providing stereoscopic images for viewing by a user of the system. The method includes providing an image or video displaying multiple spectrums of light.

### RELATED APPLICATION

This application claims priority to U.S. Provisional Application No. 63/345,800, filed May 25, 2022, the entire contents of which are incorporated herein by reference.

### BACKGROUND OF THE INVENTION

Laparoscopes or other minimally invasive surgical instruments or systems, for example, robotic surgical systems are used in a number of surgical procedures. Often these devices and systems may include additional components such as cameras. Said cameras may be paired with one or more light sources depending upon the imaging desired.

Fluorescent based imaging provides surgeons visualization of anatomy and tissue activity not visible through normal visualization. One of the most common forms used in surgery uses the dye indocyanine green (ICG) which is injected into a patient's bloodstream to image anatomical features and conditions such as tissue perfusion and blood flow. Multiple other dyes and autofluorescent capabilities all allow potential different visualizations behaviors that could aid surgeons in targeting the correct tissue to dissect.

### SUMMARY OF THE INVENTION

The present disclosure provides a multispectral camera assembly that may be employed as part of a laparoscope or surgical robotic system, and methods of use whereby an operator of a laparoscope or surgical robotic system (e.g., a surgeon) may observe an interior cavity of a subject (e.g., patient) by utilizing multispectral imaging. In some embodiments, a multispectral camera assembly enables simultaneous imaging of non-visible light, for example, fluoresce and visible light visualization of an internal body space.

The present disclosure is directed to a camera assembly configured for simultaneous multispectral imaging. According to some embodiments, the camera assembly includes a first lens assembly, a second lens assembly, a first plurality of light emitting diodes (LEDs) configured to emit light in a first wavelength range, a second plurality of LEDs configured to emit light in a second wavelength range, a plurality of LED bandpass filters, a respective one of the plurality of LED bandpass filters situated in front of each of the second plurality of LEDs to filter light emitted therefrom, a plurality of image sensors, a first of the plurality of image sensors positioned behind the first lens assembly to capture light therefrom, and a second of the plurality of image sensors positioned behind the second lens assembly to capture light therefrom, a plurality of notch filters, each notch filter situated between a respective one of the plurality of image sensors and either the first lens assembly and the second lens assembly, each notch filter configured to filter out light in a selected wavelength range transmitted by the respective first and second lens assembly; and a circuit board electronically coupled to the first and second plurality of LEDs, and the plurality of image sensors, the circuit board configured to strobe the plurality of LEDs such that each of the plurality of image sensors captures multiple spectrums of light simultaneously.

In some embodiments, the camera assembly further includes a laser. In further embodiments, the camera assembly further includes a laser bandpass filter situated adjacent to the laser to allow a selected wavelength band of light form the laser pass therethrough.

In some embodiments, the first plurality of LEDs is configured to emit light in a range from 400 nm to 700 nm and the second plurality of LEDs is configured to emit light in a range from 800 nm to 820 nm. In some embodiments, the camera assembly further includes a third plurality of LEDs configured to emit light in a range from 475 nm to 505 nm. In some embodiments, at least one of the plurality of LED bandpass filters is configured to block all light except at a wavelength around 490 nm.

In some embodiments, the second plurality of LEDs is configured to excite a dye in biological tissue. In further embodiments, the dye is fluorescein dye. In some embodiments, at least one of the plurality of LED bandpass filters is configured to allow passage of visible light.

The present disclosure is also directed to a surgical robotic system including a first camera assembly having one or more LEDs, one or more lens, one or more filter elements and one or more imaging sensors, and a second camera assembly having one or more LEDs, one or more lens, one or more filter elements and one or more imaging sensors, the first and second camera assembly providing stereoscopic images for viewing by a user of the system, a memory storing one or more instructions, and a processor configured to or programmed to read the one or more instructions stored in the memory, the processor operationally coupled to the first camera assembly and the second camera assembly to capture multiple spectrums of light simultaneously from the first and the second camera assembly.

In some embodiments, the system further includes a display operably connected to the first camera assembly and the second camera assembly, the display configured to depict an image captured by the one or more imaging sensors of each camera assembly. In further embodiments, the processor is configured to strobe the plurality of LEDs such that the image is made up of multiple spectrums of light.

In some embodiments, at least one of the first camera assembly or the second camera assembly further includes a laser. In further embodiments, at least one of the first camera assembly or the second camera assembly further includes a laser bandpass filter situated adjacent to the laser to allow a selected wavelength band of light from the laser pass therethrough.

In some embodiments, the one or more LEDs of at least one of the first camera assembly or the second camera assembly includes at least one LED configured to emit light in a range from 400 nm to 700 nm and at least one LED configured to emit light in a range from 800 nm to 820 nm. In further embodiments, the one or more LEDs of at least one of the first camera assembly or the second camera assembly further includes at least one LED configured to emit light in a range from 475 nm to 505 nm.

In some embodiments, the one or more filter elements of at least one of the first camera assembly or the second camera assembly are configured to block all light except at a wavelength around 490 nm. In some embodiments, the one or more LEDs of at least one of the first camera assembly or the second camera assembly is configured to excite a dye in biological tissue. In further embodiments, the dye is fluorescein dye. In some embodiments, the one or more filter elements of at least one of the first camera assembly or the second camera assembly are configured to allow passage of visible light.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are employed. In the accompanying drawings, like reference numbers are used to identify like components, which may not be identical components.

FIG. 1 schematically depicts an example surgical robotic system in accordance with some embodiments.

FIG. 2A is an example perspective view of a patient cart including a robotic support system coupled to a robotic subsystem of the surgical robotic system in accordance with some embodiments.

FIG. 2B is an example perspective view of an example operator console of a surgical robotic system of the present disclosure in accordance with some embodiments.

FIG. 3A schematically depicts an example side view of a surgical robotic system performing a surgery within an internal cavity of a subject in accordance with some embodiments.

FIG. 3B schematically depicts an example top view of the surgical robotic system performing the surgery within the internal cavity of the subject of FIG. 3A in accordance with some embodiments.

FIG. 4A is an example perspective view of a single robotic arm subsystem in accordance with some embodiments.

FIG. 4B is an example perspective side view of a single robotic arm of the single robotic arm subsystem of FIG. 4A in accordance with some embodiments.

FIG. 5 is an example perspective front view of a camera assembly and a robotic arm assembly in accordance with some embodiments.

FIG. 6 depicts a laparoscopic system including a laparoscope with a camera assembly, according to some embodiments.

FIG. 7 depicts components of a laparoscopic system in accordance with some embodiments.

FIGS. 8A and 8B depict a camera assembly in accordance with some embodiments.

FIG. 9 depicts a side view of a camera assembly in accordance with some embodiments.

FIGS. 10A, 10B, and 10C depict exemplary positioning or movement of a camera assembly in accordance with some embodiments.

FIG. 11 depicts a side view of the laparoscope of FIG. 8 with the cassette cover and housing cover transparent for illustrative purposes in accordance with some embodiments.

FIG. 12A depicts a side view of the motor unit and cassette of the camera assembly of FIG. 9.

FIG. 12B depicts an exploded side view of the motor unit and cassette of the camera assembly of FIG. 9 in accordance with some embodiments.

FIG. 13A depicts an internal back view of the cassette of the camera assembly of FIG. 9 in accordance with some embodiments.

FIG. 13B depicts a back view of the cassette of the camera assembly of FIG. 9 in accordance with some embodiments.

FIG. 13C depicts a transparent back view of the cassette of the camera assembly of FIG. 9 in accordance with some embodiments.

FIG. 14 depicts an exemplary 360-degree field of view of the camera assembly of FIG. 6.

FIG. 15A depicts a perspective view of a multispectral camera assembly in accordance with some embodiments.

FIG. 15B depicts a front view of the multispectral camera assembly of FIG. 15A.

FIG. 16 depicts an exploded view of the multispectral camera assembly of FIG. 15A.

FIG. 17 graphically depicts excitation-emission spectra of indocyanine green in whole blood.

FIG. 18 graphically depicts a spectral diagram of fluorescence imaging configuration for two dyes, fluorescein and indocyanine green.

FIG. 19 depicts a side view of light rays (1) collected over a wide angle by a camera assembly in accordance with some embodiments.

FIG. 20 depicts a spectral plot of changes in a filter blocking/transmittance characteristic with changes in the incident angle in accordance with some embodiments.

FIG. 21 graphically depicts the transmissivity of blue light relative to the angle from the center of the filter.

FIGS. 22B, 22C, and 22D depict channel images of a white target illuminated by white light in accordance with some embodiments. FIG. 22A shows the line profile along the yellow line of the blue channel image of FIG. 22D.

FIGS. 23B, 23C, and 23D depict channel images of a white target illuminated by white light and violet light in accordance with some embodiments. FIG. 23A shows the line profile along the yellow line of the blue channel image of FIG. 23D.

FIG. 24 depicts the integration time and operation of frame one and frame two of illustrative camera shutters.

FIG. 25 depicts a pattern of a normal, semi-exposed, spectral, and semi-exposed image sequence in accordance with some embodiments.

FIG. 26 depicts a flowchart of image processing to display a multispectral video in accordance with some embodiments.

FIGS. 27A-27D depict images and histograms associated with color correction in accordance with some embodiments.

FIG. 28 depicts an exemplary flat field image in accordance with some embodiments.

FIG. 29A depicts a color frame with no overlay in accordance with some embodiments.

FIG. 29B depicts a color frame having a fluorescein image overlaid on top of the frame in accordance with some embodiments.

FIGS. 30A-30C depict an interlaced frame between a normal and a spectral frame in accordance with some embodiments.

FIG. 31 depicts the result of combining two interlaced and one normal frame between two spectral frames of the same light source in accordance with some embodiments.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It may be understood that various alternatives to the embodiments of the invention described herein may be employed.

As used in the specification and claims, the singular form “a,” “an,” and “the” include plural references unless the context clearly dictates otherwise.

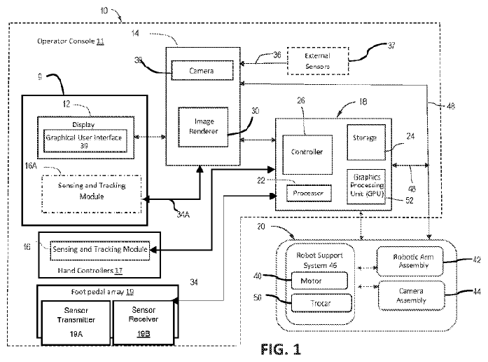

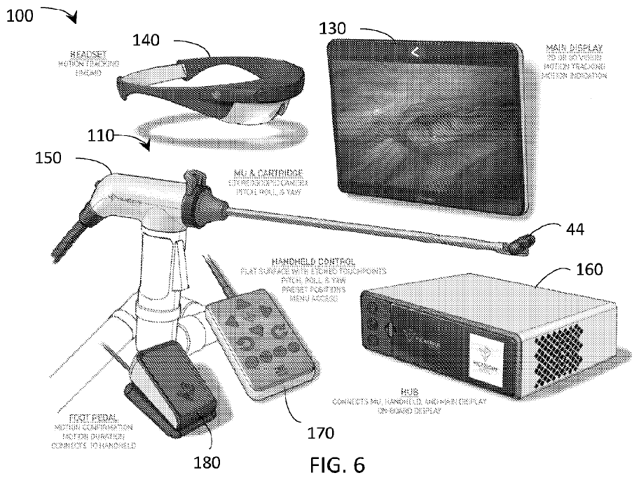

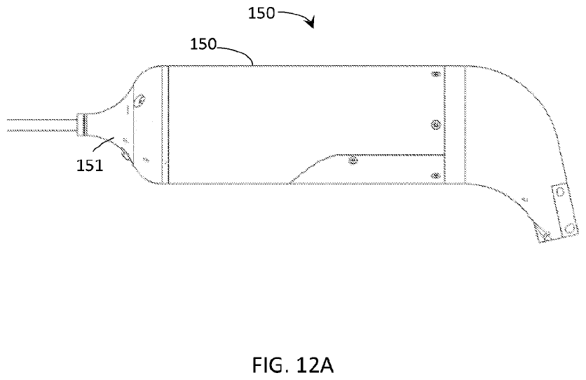

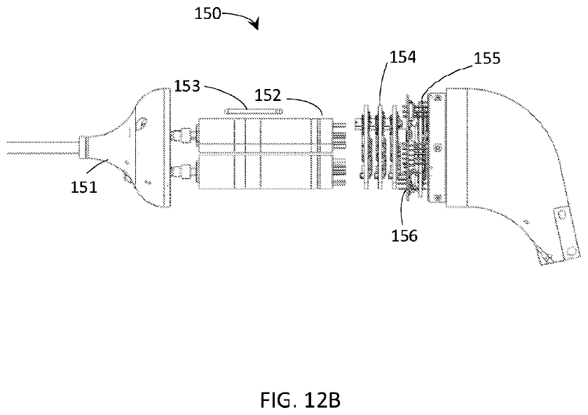

Prior to providing additional specific descriptions of the multispectral camera assembly as taught herein with respect to FIGS. 6-31, a surgical robotic system in which some embodiments could be employed is described below with respect to FIGS. 1-5. In some embodiments, the multispectral camera assembly may be employed without the surgical robotic system.

One of the challenges with designing a camera system that allows for simultaneous visualization of different spectrums (for example showing fluorescence and visible light) is the image sensor. Prior solutions addressed this problem by placing multiple different sensors in the camera system, each specific to a subset of the wavelengths selected. Another common approach is changing the Bayer pattern by adding a specific pixel that is sensitive to a subset of the bands (an IR pixel) or using hyperspectral imaging sensors with unique custom patterns. However, these approaches increase the complexity and cost of the camera systems, which may be impractical for surgical solutions. These approaches also reduce the sensitivity of the captured color spectrum because the approaches reduce the active area of imaging.

Fluorescence can help visualize blood vessels, ureters, cancer, nerves, tissue perfusion, for example. All types of fluorescence like dye, autofluorescence, and other types of differential visualization may be paired with a multispectral imaging system. The disclosed imaging system works by controlling the lighting environment and synchronizing a light source to a specific image and selectively displaying that image to the surgeon. This allows for multiple different visualizations to be used at the same time with live color for overlays without requiring additional sensors. The system employs filters on a camera assembly that selectively block specific frequencies of the emitted light.

The present disclosure provides a multispectral camera assembly whereby an operator of the camera assembly (e.g., a surgeon) may observe an interior cavity of a subject (e.g., patient) by utilizing coordinated motion of the camera assembly in accordance with some embodiments. In some embodiments, a multispectral camera assembly enables simultaneous imaging of non-visible light, for example, fluoresce and visible light visualization of an internal body space. In some embodiments, the camera assembly provides a 360-degree field of visualization, or at least two degrees of freedom for changing an orientation of a direction of view of the camera assembly without requiring a change in position (e.g., translation) or a change in orientation (e.g., tilt) of a support for the camera assembly extending external to the subject's body. In some embodiments, the camera assembly provides at least three degrees of freedom for changing the orientation of the direction of view of the camera assembly without requiring a change in position (e.g., translation) or a change in orientation (e.g., tilt) of the support for the camera assembly extending external to the subject's body. In some embodiments, the orientation of the direction of view of the camera assembly can be tilted or rotated about three orthogonal axis without translating or tilting a support for the camera assembly extending external to the subject's body.

In the following description, numerous specific details are set forth regarding the system and method of the present disclosure and the environment in which the system and method may operate, in order to provide a thorough understanding of the disclosed subject matter. It will be apparent to one skilled in the art, however, that the disclosed subject matter may be practiced without such specific details, and that certain features, which are well known in the art, are not described in detail in order to avoid complication and enhance clarity of the disclosed subject matter. In addition, it will be understood that any examples provided below are merely illustrative and are not to be construed in a limiting manner, and that it is contemplated by the present inventors that other systems, apparatuses, and/or methods can be employed to implement or complement the teachings of the present disclosure and are deemed to be within the scope of the present disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

While the camera assembly and method of the present disclosure can be designed for use with one or more surgical robotic systems, the surgical robotic system of the present disclosure can also be employed in connection with any type of surgical system, including for example robotic surgical systems, straight-stick type surgical systems, virtual reality surgical systems, and laparoscopic systems. Additionally, the camera assembly of the present disclosure may be used in other non-surgical systems, where a user requires access to a myriad of information, while controlling a device or apparatus.

The camera assembly of the present disclosure assists the surgeon in controlling movement of a robotic unit during surgery in which the robotic unit is operable within a patient. The imaging features of the present disclosure thus enable the surgeon to minimize the risk of accidental injury to the patient during surgery.

Like numerical identifiers are used throughout the figures to refer to the same elements.

FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The surgical robotic system 10 of the present disclosure employs a robotic subsystem 20 that includes a robotic unit 50 that can be inserted into a patient via a trocar through a single incision point or site. The robotic unit 50 is small enough to be deployed in vivo at the surgical site and is sufficiently maneuverable when inserted within the patient to be able to move within the body to perform various surgical procedures at multiple different points or sites. The robotic unit 50 includes multiple separate robotic arms 42 that are deployable within the patient along different or separate axes. Further, a surgical camera assembly 44 can also be deployed along a separate axis and forms part of the robotic unit 50. Thus, the robotic unit 50 employs multiple different components, such as a pair of robotic arms and a surgical or robotic camera assembly, each of which are deployable along different axes and are separately manipulatable, maneuverable, and movable. Notably, the robotic unit 50 is not limited to the robotic arms and camera assembly described herein and additional components may be included in the robotic unit. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be utilized, including but not limited to robotic surgical instruments, as well as other surgical instruments known in the art.

The operator console 11 includes a display 12, an image computing module 14, which may be a three-dimensional (3D) computing module, hand controllers 17 having a sensing and tracking module 16, and a computing module 18. Additionally, the operator console 11 may include a foot pedal array 19 including a plurality of pedals. The image computing module 14 can include a graphical user interface 39. The graphical user interface 39, the controller 26 or the image renderer 30, or both, may render one or more images or one or more graphical user interface elements on the graphical user interface 39. For example, a pillar box associated with a mode of operating the surgical robotic system 10, or any of the various components of the surgical robotic system 10, can be rendered on the graphical user interface 39. Also live video footage captured by a camera assembly 44 can also be rendered by the controller 26 or the image renderer 30 on the graphical user interface 39.

The operator console 11 can include a visualization system 9 that includes a display 12 which may be any selected type of display for displaying information, images or video generated by the image computing module 14, the computing module 18, and/or the robotic subsystem 20. The display 12 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The display 12 can also include an optional sensing and tracking module 16A. In some embodiments, the display 12 can include an image display for outputting an image from a camera assembly 44 of the robotic subsystem 20.

The hand controllers 17 are configured to sense a movement of the operator's hands and/or arms to manipulate the surgical robotic system 10. The hand controllers 17 can include the sensing and tracking module 16, circuitry, and/or other hardware. The sensing and tracking module 16 can include one or more sensors or detectors that sense movements of the operator's hands. In some embodiments, the one or more sensors or detectors that sense movements of the operator's hands are disposed in the hand controllers 17 that are grasped by or engaged by hands of the operator. In some embodiments, the one or more sensors or detectors that sense movements of the operator's hands are coupled to the hands and/or arms of the operator. For example, the sensors of the sensing and tracking module 16 can be coupled to a region of the hand and/or the arm, such as the fingers, the wrist region, the elbow region, and/or the shoulder region. Additional sensors can also be coupled to a head and/or neck region of the operator in some embodiments. In some embodiments, the sensing and tracking module 16 can be external and coupled to the hand controllers 17 via electricity components and/or mounting hardware. In some embodiments, the optional sensor and tracking module 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensing and tracking module 16 can employ sensors coupled to the torso of the operator or any other body part. In some embodiments, the sensing and tracking module 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensing and tracking module 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computing module 18 and hence employed by the surgical robotic system 10.

The sensors generate position and/or orientation data indicative of the position and/or orientation of the operator's hands and/or arms. The sensing and tracking modules 16 and/or 16A can be utilized to control movement (e.g., changing a position and/or an orientation) of the camera assembly 44 and robotic arms 42 of the robotic subsystem 20. The tracking and position data 34 generated by the sensing and tracking module 16 can be conveyed to the computing module 18 for processing by at least one processor 22.

The computing module 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arms 42, or both. In some embodiments, the controller 26 can also adjust the pan and tilt of the camera assembly 44 to follow the movement of the operator's head. The computing module may further include a graphics processing unit (GPU) 52, discussed in further detail below.

The robotic subsystem 20 can include a robot support system (RSS) 46 having a motor 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms assembly 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arms assembly 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computing module 18. The motor 40 can thus generate signals for controlling one or more motors that in turn can control and drive the robotic arms 42, including for example the position and orientation of each robot joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 300 through the trocar 50.

The trocar 50 is a medical device that can be made up of an awl (which may be a metal or plastic sharpened or non-bladed tip), a cannula (essentially a hollow tube), and a seal in some embodiments. The trocar 50 can be used to place at least a portion of the robotic subsystem 20 in an interior cavity of a subject (e.g., a patient) and can withdraw gas and/or fluid from a body cavity. The robotic subsystem 20 can be inserted through the trocar 50 to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensing and tracking module 16, the robotic arms 42, the camera assembly 44, and the like), and for generating control signals in response thereto. The motor 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic arms assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers 17 grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes an HMD, the display can include the built-in sensing and tracking module 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracking module. In some embodiments, the sensing and tracking module 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensing and tracking module 16A for tracking at least a portion of the operator's head.

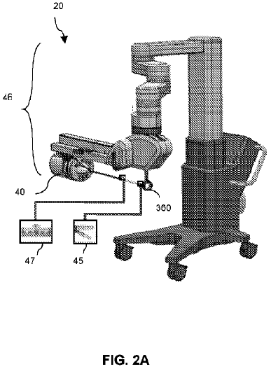

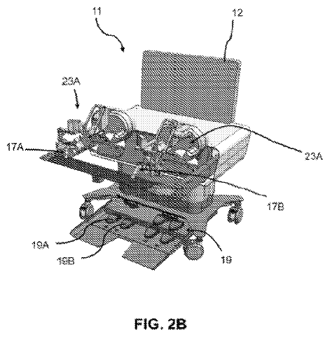

FIG. 2A depicts an example robotic arms assembly 20, which is also referred to herein as a robotic subsystem, of a surgical robotic system 10 incorporated into or mounted onto a mobile patient cart in accordance with some embodiments. In some embodiments, the robotic arms assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes a display 12, hand controllers 17, and also includes one or more additional controllers, such as a foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

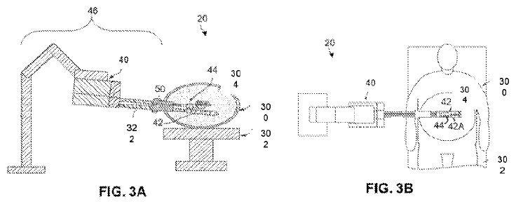

FIG. 3A schematically depicts a side view of the surgical robotic system 10 performing a surgery within an internal cavity 304 of a subject 300 in accordance with some embodiments and for some surgical procedures. FIG. 3B schematically depicts a top view of the surgical robotic system 10 performing the surgery within the internal cavity 304 of the subject 300. The subject 300 (e.g., a patient) is placed on an operation table 302 (e.g., a surgical table 302). In some embodiments, and for some surgical procedures, an incision is made in the patient 300 to gain access to the internal cavity 304. The trocar 50 is then inserted into the patient 300 at a selected location to provide access to the internal cavity 304 or operation site. The RSS 46 can then be maneuvered into position over the patient 300 and the trocar 50. In some embodiments, the RSS 46 includes a trocar mount that attaches to the trocar 50. The robotic arms assembly 20 can be coupled to the motor 40 and at least a portion of the robotic arms assembly can be inserted into the trocar 50 and hence into the internal cavity 104 of the patient 300. For example, the camera assembly 44 and the robotic arm assembly 42 can be inserted individually and sequentially into the patient 300 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 300, thus reducing the trauma experienced by the patient 300. In some embodiments, the camera assembly 44 and the robotic arm assembly 42 can be inserted in any order or in a specific order. In some embodiments, the camera assembly 44 can be followed by a first robotic arm of the robotic arm assembly 42 and then followed by a second robotic arm of the robotic arm assembly 42 all of which can be inserted into the trocar 50 and hence into the internal cavity 304. Once inserted into the patient 300, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

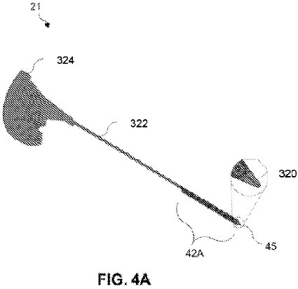

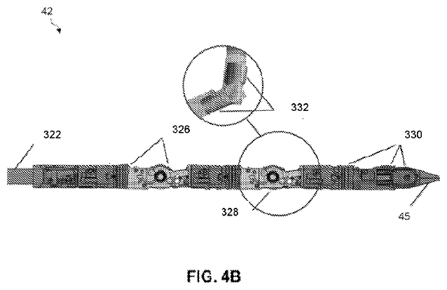

FIG. 4A is a perspective view of a robotic arm subassembly 21 in accordance with some embodiments. The robotic arm subassembly 21 includes a robotic arm 42A, the end-effector 45 having an instrument tip 320 (e.g., monopolar scissors, needle driver/holder, bipolar grasper, or any other appropriate tool), a shaft 322 supporting the robotic arm 42A. A distal end of the shaft 322 is coupled to the robotic arm 42A, and a proximal end of the shaft 322 is coupled to a housing 324 of the motor 40 (as shown in FIG. 2A). At least a portion of the shaft 322 can be external to the internal cavity 304 (as shown in FIGS. 3A and 3B). At least a portion of the shaft 322 can be inserted into the internal cavity 304 (as shown in FIGS. 3A and 3B).

FIG. 4B is a side view of the robotic arm assembly 42. The robotic arm assembly 42 includes a virtual shoulder 326, a virtual elbow 328 having position sensors 332 (e.g., capacitive proximity sensors), a virtual wrist 330, and the end-effector 45 in accordance with some embodiments. The virtual shoulder 326, the virtual elbow 328, the virtual wrist 330 can include a series of hinge and rotary joints to provide each arm with positionable, seven degrees of freedom, along with one additional grasping degree of freedom for the end-effector 45 in some embodiments.

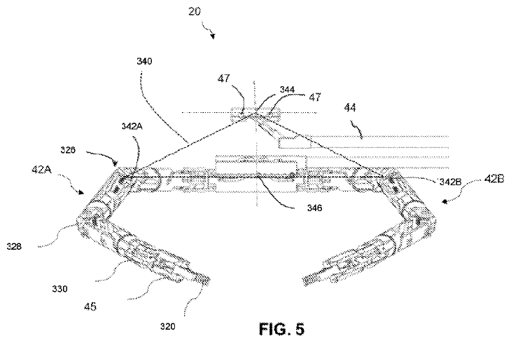

FIG. 5 illustrates a perspective front view of a portion of the robotic arms assembly 20 configured for insertion into an internal body cavity of a patient. The robotic arms assembly 20 includes a robotic arm 42A and a robotic arm 42B. The two robotic arms 42A and 42B can define, or at least partially define, a virtual chest 340 of the robotic arms assembly 20 in some embodiments. In some embodiments, the virtual chest 340 (depicted as a triangle with dotted lines) can be defined by a chest plane extending between a first pivot point 342A of a most proximal joint of the robotic arm 42A (e.g., a shoulder joint 326), a second pivot point 342B of a most proximal joint of the robotic arm 42B, and a camera imaging center point 344 of the camera(s) 47. A pivot center 346 of the virtual chest 340 lies in the middle of the virtual chest.

In some embodiments, sensors in one or both of the robotic arm 42A and the robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, a camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

Hand controllers for a surgical robotic system as described herein can be employed with any of the surgical robotic systems described above or any other suitable surgical robotic system. Further, some embodiments of hand controllers described herein may be employed with semi-robotic endoscopic surgical systems that are only robotic in part.

As explained above, controllers for a surgical robotic system may desirably feature sufficient inputs to provide control of the system, an ergonomic design and “natural” feel in use.

In some embodiments described herein, reference is made to a left hand controller and a corresponding left robotic arm, which may be a first robotic arm, and to a right hand controller and a corresponding right robotic arm, which may be a second robotic arm. In some embodiments, a robotic arm considered a left robotic arm and a robotic arm considered a right robotic arm may change due a configuration of the robotic arms and the camera assembly being adjusted such that the second robotic arm corresponds to a left robotic arm with respect to a view provided by the camera assembly and the first robotic arm corresponds to a right robotic arm with respect view provided by the camera assembly. In some embodiments, the surgical robotic system changes which robotic arm is identified as corresponding to the left hand controller and which robotic arm is identified as corresponding to the right hand controller during use. In some embodiments, at least one hand controller includes one or more operator input devices to provide one or more inputs for additional control of a robotic assembly. In some embodiments, the one or more operator input devices receive one or more operators inputs for at least one of: engaging a scanning mode, resetting a camera assembly orientation and position to a align a view of the camera assembly to the instrument tips and to the chest; displaying a menu, traversing a menu or highlighting options or items for selection and selecting an item or option, selecting and adjusting an elbow position, and engaging a clutch associated with an individual hand controller.

In some embodiments, additional functions may be accessed via the menu, for example, selecting a level of a grasper force (e.g., high/low), selecting an insertion mode, an extraction mode, or an exchange mode, adjusting a focus, lighting, or a gain, camera cleaning, motion scaling, rotation of camera to enable looking down, etc.

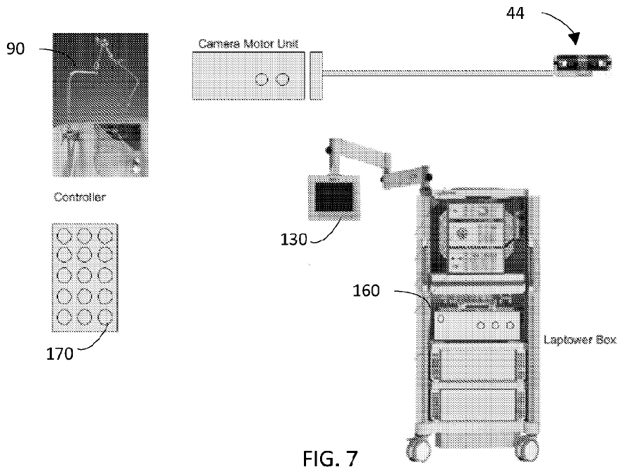

FIGS. 6 and 7 depict a system 100 including a laparoscope 110 with a camera assembly 44 in according to some embodiments. In some embodiments, the laparoscope 110 is configured to be hand held. In some embodiments, the laparoscope 110 is configured to be held by, mounted to, and/or mated with an external support. The system 100 can include some or all of the components that make up the system 10 depending on the type of surgical procedure. As described herein, the system 100 represent a laparoscope embodiments, but is not limited thereto.

In some embodiments, the system 100 also includes a display for displaying images or image data (e.g., a camera feed, a processed camera feed, images generated from a processed camera feed or sensor feed, images generated from two-dimensional sensor data) obtained from the camera assembly 44. In some embodiments, the display is or includes a display screen 130, such as a monitor or tablet. In some embodiments, the display includes a two-dimensional (2D) display and/or a three-dimensional (3D) display In some embodiments, the display may include a virtual reality (VR) or augmented reality (AR) headset or a different form of a VR or AR device (e.g., smart glasses, heads up displays (HUDs), holographic displays, etc.). In some embodiments, the system 100 includes a motion-tracking headset 140 or other head movement sensing device, system, or mechanism. In some embodiments, the motion-tracking headset is also used as a display or a component of a display. In some embodiments, output regarding a motion of an operator's head from the a motion tracking headset 140 is used an input to control an orientation of a direction of view of the camera assembly 44 as described in further detail below. In some embodiments, use of a motion tracking headset 140, and/or an AR headset allows an operator to change a direction of view of the camera assembly 44 while retaining vision of surgical tools. Some examples and aspects of incorporation of camera feeds into a VR/AR headset during surgery are described in U.S. Pat. No. 10,285,765, which is hereby incorporated by reference in its entirety. Some exemplary display screens are described in International Publication No. WO 2021/092194, which is hereby incorporated by reference in its entirety. In some embodiments, images are presented on the display 130 at sixty frames per second. In other embodiments, the images are presented on the display 130 at one hundred and twenty frames per second.

In some embodiments, the system 100 also includes a motor unit 150 that drives motion of at least a portion of the camera assembly 44. In some embodiments, the motor unit 150 can be implemented as the motor 40 in the system 10 to drive motion of at least a portion of the camera assembly 44 as taught herein. In some embodiments, the system 100 includes a control and processing unit or system (e.g., a laptower box 160) for receiving input from operator controllers and for controlling motion of the camera assembly 44. In some embodiments, the control and processing unit or control and processing system also generates output for the display (e.g., display screen 130). In some embodiments, the control and processing unit or control and processing system may be disposed in a mobile cart, tower, box, or laptower box.

In some embodiments, the system 100 also includes one or more operator controllers to receive input for controlling the laparoscope 110. In some embodiments, the one or more operator controllers also control the image or data displayed. In some embodiments, the operator controllers include any of a foot pedal 180, a handheld controller 170, and a motion-tracking headset 140. In some embodiments, other or additional operator controllers may be employed. The laparoscope 110 and camera assembly 44 may be controlled by operator input provided via a motion tracking headset 140, a foot pedal 180, a handheld controller 170, one or more buttons on the motor unit 150 or a combination thereof (see FIGS. 6 and 7). In some embodiments, the system 100 may further include a holder 190 for the motor unit 150. The holder 190 may be a stable unit configured to hold the motor unit 150 in a stationary position without support from the operator or assistant. An exemplary holder 190 is the Iron Intern® produced by Automated Medical Products Corp.

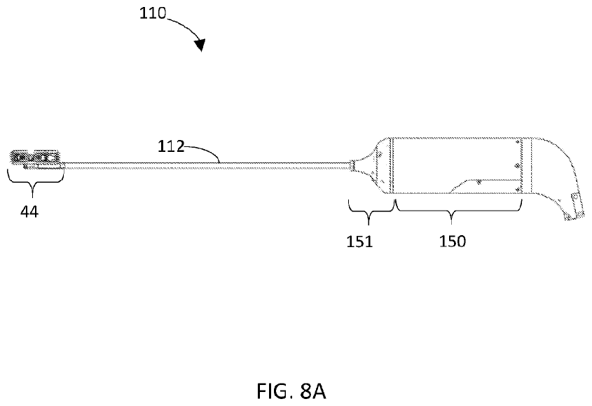

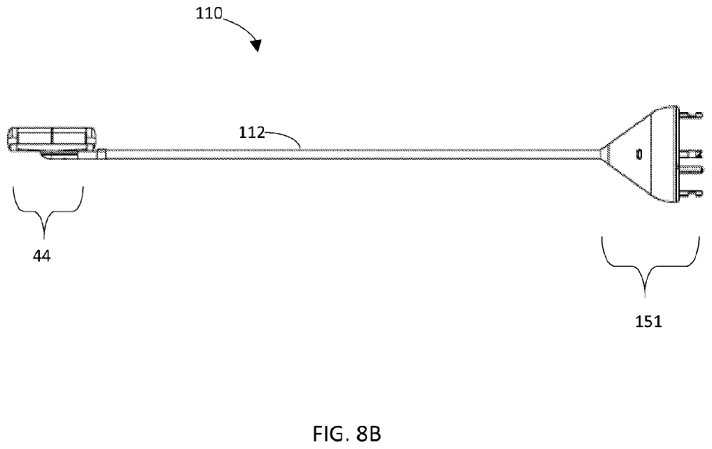

FIGS. 8A and 8B depict opposing side views of a laparoscope 110 including a camera assembly 44 in accordance with some embodiments. The camera assembly 44 is disposed at a distal end of the laparoscope 110. The camera assembly 44 is supported by a support 112 (e.g., a support tube) in some embodiments. The support 112 connects with the motor unit 150 of the laparoscope 44 in some embodiments. The motor unit 150 may be configured to be handheld. The motor unit 150 may be configured to be held by, mounted to, connected to and/or mated with an external support or holder in accordance with some embodiments. In some embodiments, the motor unit 150 is sized and weighted similarly to a soda can. In some embodiments, a cassette 151 connects the support tube 112 with the motor unit 150 and connects elements or components extending through the support tube 112 with the motor unit 150.

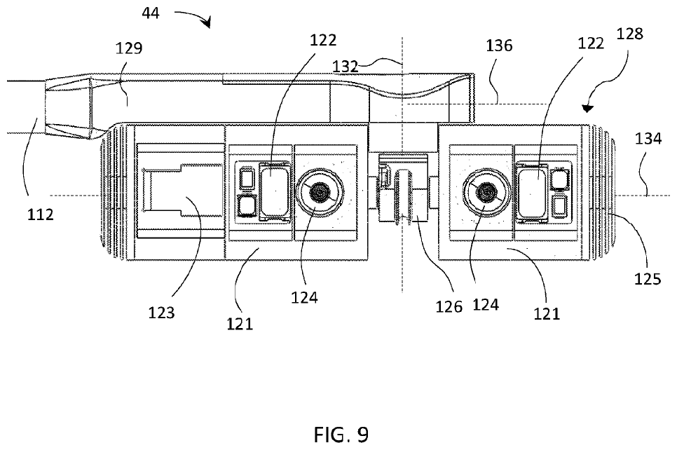

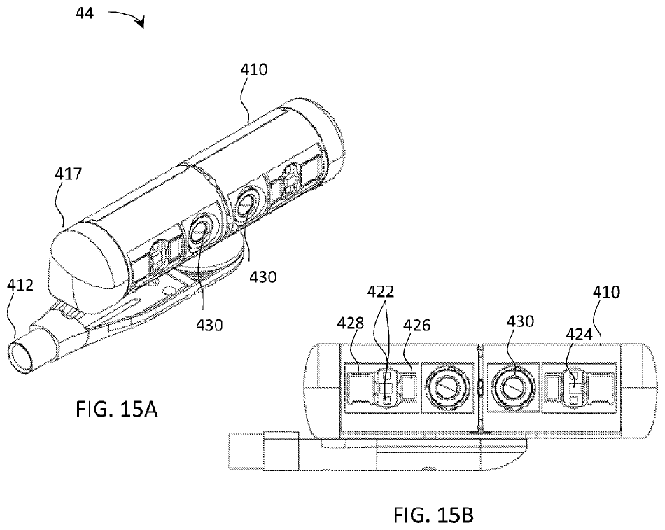

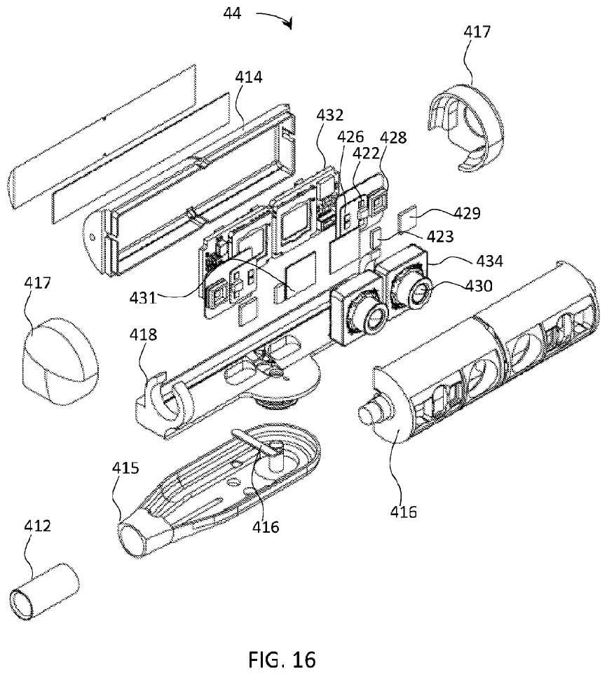

FIG. 9 depicts a side view of a distal end of the laparoscope 110 including the camera assembly 44 and the support 112. In some embodiments, the camera assembly 44 includes a camera unit that includes one or more camera modules 124 and/or other imaging modules. The camera assembly may have more than one camera unit in some embodiments. In some embodiments, a camera unit 128 includes an image sensor mount 121 for mounting image sensors and/or camera modules 124. In some embodiments, multiple camera modules 124 are used to generate stereoscopic images. In some embodiments, the camera unit 128 may include multiple camera or imaging modules for imaging different types of light. In some embodiments, the camera unit may include sensors or detectors that sense or detect nonvisible electromagnetic signals. In some embodiments, the camera unit includes one or more light sources (e.g., one or more light emitting diodes (LEDs) 122, which may include a combination of any of LEDs 422, 424, or 426 as discussed in further detail below). In some embodiments, the camera unit 128 includes light sources that produce light with different spectra or in different spectral bands. Such light sources are discussed in further detail below with regards to FIGS. 15A, 15B, and 16. In some embodiments, the camera unit includes one or more light sources that produce light outside the visible light spectrum (e.g., a light source that produces infrared (IR) light, and/or a light source that produce ultraviolet (UV) light). The camera unit 128 field of view may be illuminated by one more light sources 122 that produce one or more types of light, such as at least one source that produces light in the visible spectrum (e.g., an LED emitting white light)) and/or at least one light source that produces light that is not in the visible spectrum (e.g., a light source that produces infrared (IR) light, and/or a light source that produce ultraviolet (UV) light).

The system 100 includes an imaging mode. In some embodiments, the system 100 is configured for more than one imaging mode that may be engaged simultaneously to produce multispectral imaging. For example, multispectral illumination with light in the visible spectrum and detection of light in the visible spectrum may produce the primary image output displayed in accordance with some embodiments. In some embodiments, the system 100 can also generate secondary image output based on illumination with light outside the visible spectrum (e.g., IR light or UV light). In some embodiments, a light spectrum used to illuminate may be different from a light spectrum detected for generation of images (e.g., for fluorescence images). In some embodiments, one or more filters (e.g., digital filters and/or physical filters as discussed below) may be employed to isolate certain wavelength ranges of interest for some images modes. In some embodiments, input from only certain color channels (e.g., red, green, and/or blue) of image detectors may be employed. A visual output from one imaging mode may be displayed overlaid on output from another imaging mod in some embodiment. For example, a non-visible spectrum image output (e.g., an IR imaging output) may be overlaid over a primary (e.g., visible light) image output on the display in some embodiments to produce a multispectral image or video. In some embodiments, a green pixel may be replaced with a broadband sensitive pixel or infrared sensitive pixel to enable better sensitivity in multi-spectral operational modes.

In some embodiments, the system 100 strobes between one type of light source and another type of light source (e.g., between the white light source and the IR light source) to produce a combined multispectral image or video. For example, the system 100 may shut off the white light source for one frame, during which the infrared light source is turned on. In some embodiments, the system 100 is configured to strobe a primary illumination source to drop a frame from the primary image feed (e.g., the visible light feed or the white light feed) multiple times per second (e.g., six times per second). In such embodiments, when the primary illumination source (e.g., the visible light source or the white light source) is not fully powered, frames are either discarded or depicted with an image from another imaging mode (e.g., an IR imaging mode). In some embodiments, the system 100 is configured to strobe more than one type of light source. For example, the system may strobe white light LEDs, blue light LEDs, or a combination of the two. One or more of the light sources may include a laser source, such as the laser discussed in further detail below.

In some embodiments, the camera assembly 44 performs autofocus. In some embodiments, the multispectral camera assembly 44 is configured to automatically focus on an area in the center of the strobed light. In some embodiments, the multispectral camera assembly 44 may provide an improved field of view and/or depth of field as compared to cameras employed with conventional laparoscopes or robotic surgical devices.

In some embodiments, a camera unit 128 may also include at least one pulsed laser light source and the system 44 may employ light detection and ranging (LIDAR) functionality. In some embodiments, LIDAR may be employed for auto-focus. In some embodiments, LIDAR may be employed for obtaining a three-dimensional representation or map of at least a portion of a body cavity. In some embodiments, a camera unit 128 may include an additional mount 123 for a LIDAR source or a dot matrix projector. In some embodiments, the camera assembly 44 may include one or more features or components for heat dissipation. For example, a camera unit 128 may include one or more heat dissipation structures 125 (e.g., fins). The camera unit 44 may also provide an improved field of view and depth of field as compared to cameras employed with conventional laparoscopes or robotic surgical devices in accordance with some embodiments.

In some embodiments, the camera assembly 44 includes a zoom capability (e.g., a 2× zoom capability) that does not reduce a resolution of the image displayed when the maximum zoom is employed. For example, in some embodiments, one or more camera modules may a higher resolution than needed for display of a full field of view. In those embodiments, only a subset of the pixels from the camera modules are displayed. In some embodiments, a zoom may be employed in which a smaller selected portion of the field of view is displayed, but a larger proportion of the pixels in the pixels in the selected portion of the field of view are displayed, resulting in a zoom that does not reduce the resolution of the image displayed.

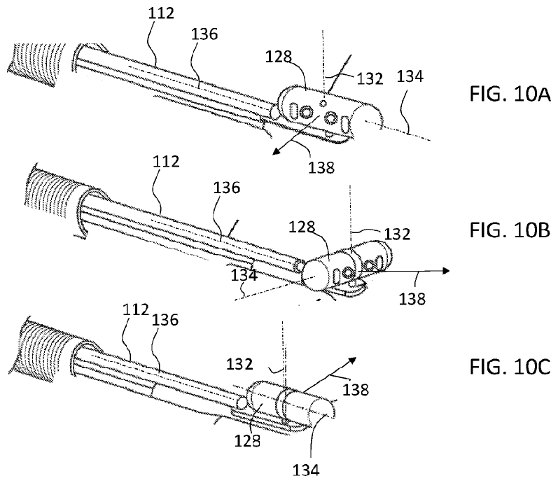

The camera assembly 44 has a yaw axis 132, a pitch axis 134, and a roll axis 136 (see FIGS. 9-10C). A direction of view of the camera unit 128 of the camera assembly 44, which may be referred to as a viewing direction 138, can be rotated or tilted about the different axes (e.g., the yaw axis 132, the pitch axis 134, and the roll axis 136), two of which are always orthogonal to each other (e.g., the yaw axis 132 and the pitch axis 134) without requiring motion of a support 112 extending external to the subject's body cavity (see FIGS. 9-10C). One or more actuators, e.g., a pitch and yaw composite actuator 132, connect camera unit 128 to a main body 129 of the camera assembly 44 (see FIG. 9). In FIGS. 9, 10A and 10C, a pitch axis 134 of the camera body 128 is parallel to a roll axis 136 of the camera assembly, which corresponds to an insertion or removal configuration. The camera unit 128 can be rolled such that the viewing direction 138 is back toward an insertion point in some embodiments. Exemplary camera assemblies may include some elements described in U.S. Pat. No. 11,583,342, which is hereby incorporated by reference in its entirety.

Conventional robotic surgical systems are configured to yaw, pitch, and roll a camera in a manner similar to an operator moving the camera assembly to view the target area. These conventional systems require motion external to the patient to manipulate a camera, and may require repositioning the system through multiple different insertion ports, to capture a 360-degree field of view of a surgical site. Therefore, some conventional systems require a large amount of extraneous movement or use of additional systems over the same surgical area, making the surgical areas more cluttered and the procedure more complicated. By comparison, the disclosed camera assembly 44 is configured to provide a 360-degree field of view without requiring movement of the camera 44 relative to a support 112 of the laparoscope 110 extending outside the subject's body cavity in accordance with some embodiments. For example, an operator will be able to insert the laparoscope 110 into a surgical area and obtain a view back toward the insertion point, such as a trocar if a trocar is used, without motion of an external support in accordance with some embodiments. Removing or reducing the required external motion reduces the forces exerted on the trocar and lessens damage to tissue surrounding the trocar.

Examples and explanations of actuators for moving one or more components of a camera assembly, a support for a camera assembly, and a motor unit to drive movement of the camera assembly appear in U.S. Pat. No. 11,583,342 which is incorporated by reference herein in its entirety. In some embodiments, the camera assembly 44 is configured to be moved for the purposes of positional correction. Movement of the camera assembly 44 may be performed as described in International Publication No. WO 2021/231402, which is hereby incorporated by reference in its entirety.

In some embodiments, the insertable portion of the laparoscope 110 has a diameter between a range of about 12 mm and 18 mm. In some embodiments, the diameter may be between 15 mm and 18 mm. In some embodiments, the insertable portion of the laparoscope 110 has a diameter of 18 mm and is insertable into a trocar having a diameter of 118 mm. In some embodiments, the insertable portion of the laparoscope 110 is inserted into a flexible trocar. A flexible trocar enables an operator to create a smaller incision port. When the insertable portion of the laparoscope 110 is inserted into a cervix, an oval trocar may be employed.

Control of the camera assembly 44 provides for improved visualization of a surgical site in accordance with some embodiments. For example, the system 100 of FIG. 6 may provide an improved field of vision during a hysterectomy, appendectomy, resection, excision, or other surgical procedure suitable for minimally invasive techniques and instruments. Further, the camera assembly 44 of FIG. 6 may be controlled directly by an operator without relying upon an assistant in accordance with some embodiments. Exemplary positioning or movement of the camera assembly 44 is depicted in FIGS. 10A, 10B, and 10C.

In some embodiments, the laparoscope 110 may be insertable into a surgical site without use of a trocar. For example, the laparoscope 110 may be inserted vaginally. Vaginal insertion may reduce the required number of 5 mm ports for a hysterectomy to two or three in total. In some embodiments, the laparoscope 110 may be insertable into a surgical site with use of a trocar, or the camera assembly 44 associated with a surgical robotic system may be insertable with use of a trocar.

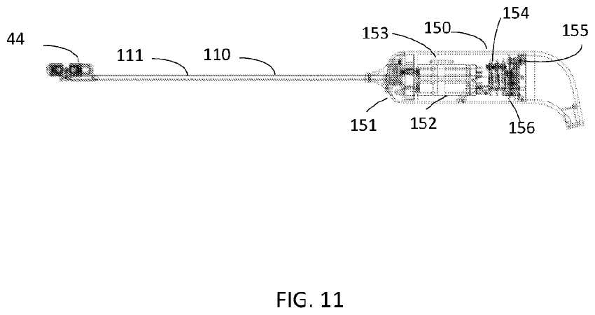

FIG. 11 depicts a side view of the laparoscope 110 including a camera assembly 44, a support 110, a cassette 151 and a motor unit 150 with a cover of the cassette 151 and a cover of the motor unit 150 transparent for illustrative purposes, in accordance with some embodiments.

The system 100 may further include a motor unit 150 configured to manipulate the camera assembly 44 with motors (e.g., mason motors) that drive one or more actuators 126 of the camera assembly 44. The motor unit 150 may include one or more motor control boards (MCBs) 156. The motor until may include one or more serializer/deserializer boards 155. The motor unit 150 may also include any of a power connector, a universal serial bus (USB) connector, and a fiber surface-mount technology (SMT) connector in accordance with some embodiments. The connectors may interface with the camera assembly 44 and/or a control and processing unit or system (e.g., a laptower box 160). In some embodiments, the motor unit 150 includes one or more inertial measurement units 153.

In some embodiments, the motor unit 150 includes at least two buttons for controlling one or more aspects of the camera assembly 44. For example, one button may be engaged to orient the camera 44 for insertion or extraction. In such an example, the other button may be engaged to lock the camera assembly 44 (i.e. with a gimbal lock) to allow an operator or assistant to move the motor unit 150 while the camera assembly 44 remains in a fixed position within a surgical site.

In some embodiments, the support 112 and elements extending within the support tube (e.g., electronic cables and mechanical actuation cables) are mated with the motor unit 150 via a cassette 151. In some embodiments, the laparoscope 110 is configured for positioning a sterile drape or cover between the cassette 151 and the motor unit 150. In some embodiments, at least a portion of the laparoscope 110 is reusable for multiple procedures. In some embodiments, at least a portion of the laparoscope 110 may be sterilized and reused for multiple procedures, for example up to ten procedures. In some embodiments, at least a portion of the laparoscope 110 may be cleaned in an autoclave. In some embodiments, at least a portion of the laparoscope 110 may be single use. In some embodiments, the cassette 151, support, 112 and camera assembly 44 are single use and the motor unit 150 is reusable. In some embodiments, the cassette 151, support, 112 and camera assembly 44 are sterilizable for reuse a limited number of times. In some embodiments cassette 151, support, 112 and camera assembly 44 are reusable for a limited number of times that is smaller than a number of times that the motor unit 150 is reusable.

In some embodiments, the motor unit 150 may be removed from the laparoscope 110 after surgery for repair or replacement.

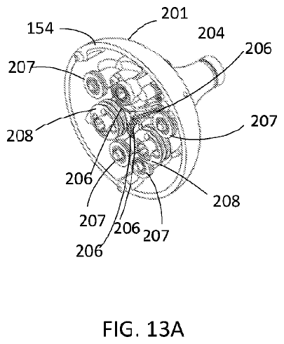

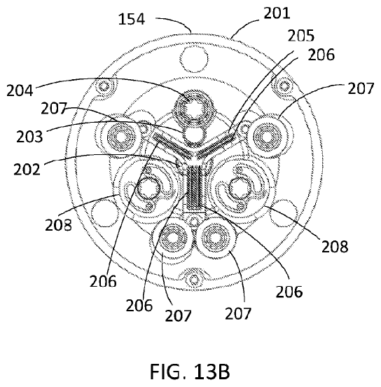

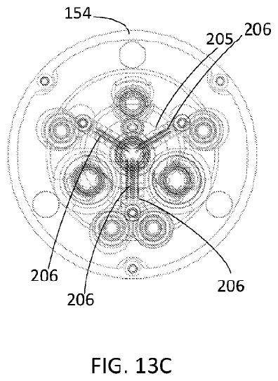

FIGS. 13A-13C are different views of the cassette 151 in accordance with some embodiments. The cassette 151 includes a cassette housing 201, a support tube gear 202 for rotating the support tube 112 for rotating the support tube, and an intermediary gear 203 in accordance with some embodiments. The cassette 151 may further include redirecting pulleys 206, and a redirecting pulley mount 205. The cassette 151 may also include a roll spooley 204, and two spooleys 208 for yaw and pitch. The cassette 151 may also include an idler pulley 207. The cassette 151 and motor unit 150 may include some elements and aspects described in U.S. Pat. No. 11,583,342, which is hereby incorporated by reference in its entirety.

The system 100 may further include a control and processing unit or system 150 (e.g., a laptower box 160 or computing module 18 as described above). The laptower box 160 may be sized to be placed on a laptower. The computing module 18 or box 160 may be configured to provide outputs to data recorders and/or the display 130 or headset 140. The computing module 18 or laptower box 160 may interface with and power any or all of the camera assembly 44, a display 12 (e.g., a VR/AR headset), and operator controllers (e.g., handheld controller 170, food pedal 180, and/or motion tracking headset). The computing module 18 or laptower box 160 may include a display 130. In some embodiments, the system 100 includes a first display 130 and a second display on the laptower box 160.

The handheld controller 170 (such as hand controller 17) may be configured to provide command interfaces for the camera assembly 44. For example, an operator or assistant may use the handheld controller 170 to move the camera unit 128, orient the camera unit 128, and/or select menu options on the display 130. In some embodiments, the handheld controller 170 may be sized and shaped similar to hand controller 17 discussed above. In some embodiments, the handheld controller 170 is connected to a foot pedal 180 (such as foot pedal array 19 discussed above), for example with a wired connection.

The system 100 may be operable in a hand-free mode in accordance with some embodiments. For example, the headset 140 may track an operator's head motion as the operator looks to the edges of a bezel of the headset 140. This head motion may trigger a modality for the operator to activate with the foot pedal 180. The operator may then increase pressure upon the pedal 180 to increase the rate of movement of the camera unit 128 toward a selected direction, as an example. When the desired movement is complete, the operator may release the foot pedal 180 and continue operating. As another example, the operator may roll the camera unit 128 by rolling their head slightly. As another example, operating in collaboration with the foot pedal 180, the surgeon is capable of, for example, tilting their head backwards while engaging the foot pedal 180 to move the camera unit 128 upwards, releasing the foot pedal 180 and resetting their head to a desired position, and then reengaging the foot pedal 180 and tilting their head backwards to move the camera unit 128 further upwards. Accordingly, the surgeon is capable of continuing to move the camera unit 128 upwards by clutching in and out of the camera mode with the foot pedal 180.

Other gestures may be programmed to perform other functions such as a long blink that signals cleaning or wiping of the camera unit 128 or rotating the camera unit 128 up to, and including, 360 degrees. In some embodiments, an operator straining one or both eyes may indicate a blurry camera feed or a headset alignment. After detecting the strain, the system 100 generates a message on the display 130 indicating the blurry feed or headset misalignment.

Head movements may be coupled to holding down the foot pedal 180, releasing the foot pedal 180, or pressing the foot pedal 180. For example, double tapping the foot pedal 180 could trigger a menu to be displayed and controlled with head motion and/or the foot pedal 180 to select additional options. As another example, leaning forward or backward may control a zoom feature of the camera assembly 44.

In some embodiments, the system 100 tracks eye movement of the operator wearing the headset 140 to determine when the operator is engaging the system 100. The eye tracking may also prompt the system 100 to identify, and focus on, a specific area of a surgical site that an operator is looking at. When focused on a specific area, the camera assembly 44 may be configured to maintain a visual of the specific area while the laparoscope 110 or camera assembly 44 is moved by an external force.

In some embodiments, an operator may look to the edges of vision of the VR/AR headset 130 to trigger different operational modes to select with the foot pedal 180. Additionally or alternatively, looking to the edge of the screen of the headset 140 may adjust the speed of movement of the camera assembly 44 or laparoscope 110 without use of a foot pedal 180. Accuracy of the eye tracking may be improved through the use of retroreflectors in the headset 140.

In some embodiments, the system 100 may include a joystick, or other multiple input device such as capacitive/inductive sensing pads, attached to the motor unit 150. The joystick may be configured to control movement of the camera unit 128, either alone or in conjunction with the headset 140 or foot pedal 180. In some embodiments, movement of the joystick or capacitive/inductive sensing pads enables fast, controlled motion without some additional form of confirmation from the headset 140 or foot pedal 180.

The system 100 may include a plurality of imaging modes. For example, a first imaging mode may be the live stream that is captured by the camera unit 128 and output to the user. A secondary imaging mode may be used to provide the surgeon with a secondary display. For example, using image overlaying, a smaller display may be output based on information captured from a variety of different sensors capable of being incorporated into the camera assembly 44.

As one example, in a secondary imaging mode using a dot projector, the camera assembly 44 may output an infrared (IR) light between each output of a color (white) light (e.g., light of different frequencies). The information captured during the output of the IR light may be output on the secondary display. The secondary display in this example thus shows the dots on the environment being captured by the camera unit 128. This feature is advantageous in providing further depth information to a surgeon. The information may also be captured to generate depth maps of the environment being captured by the camera unit 128. Methods of determining depth perception in vivo are discussed in International Publication No. WO 2021/159048, which is hereby incorporated by reference in its entirety.

As another example, the camera unit 128 may provide spectral imaging to detect specific bodily features, for example a ureter or bladder. The system 100 may be configured to map bodily features to detect and outline organs on the display. For example, the camera assembly 44 may be configured to perform indocyanine green (ICG) imaging and may detect a ureter treated with dyes such as methylene blue, UreterBlue, or ZW800-1. The camera unit 128 may be configured to identify cancer tissue dyed with fluorescein. The camera unit 128 may be configured to identify nerve vascularization dyed with GE3111.

In some embodiments, the system 100 is configured to digitally tag identified tissue to monitor movement of the tissue during a procedure. For example, the system 100 may identify the edge of a bladder and monitor movement of the bladder during a hysterectomy. Detecting and monitoring the edge of the bladder may inform an operator as to where to make incisions for the hysterectomy. As another example, the system may identify cancer tissue of nerve tissue dyed with an appropriate dye. By identifying tissues within the surgical site, the surgeon is also able to distinguish between different tissues and determine, for example, which tissues to avoid contact with during surgical procedure.

In some embodiments, an operator or assistant may measure a distance between two points within the field of view of the camera assembly 44 by identifying the points with a grasper of the laparoscope 110 or a robotic arm, or identifying the points on the display 130 or headset 140. The system 100 may be configured to then measure the distance between the identified points and depict the calculated distance on the display 130 or headset 140.

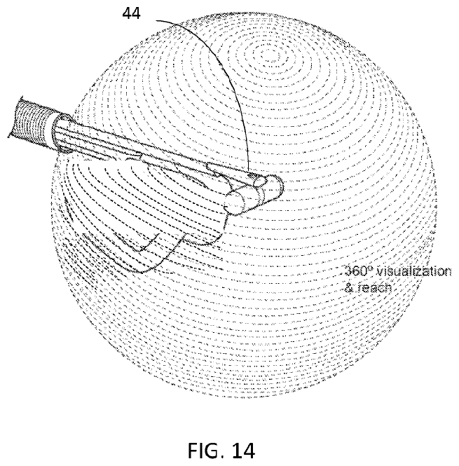

In some embodiments, the operator or assistant can prompt the system 100 to capture a 360-degree scan of the surgical site. To capture the scan, the camera assembly 44 may be configured to spin in place (e.g., via roll about the roll axis). The collected image data may be generated as a visual mesh that can be viewed on the display 130 or headset 140 during the procedure or after the surgical procedure is completed. An exemplary 360-degree field of visualization is depicted in FIG. 14.

FIG. 15A depicts a perspective view of a multispectral camera assembly 44 in accordance with some embodiments. The multispectral camera assembly 44 may include a housing 410 containing multiple LEDs 422, 424, 426. In some embodiments, the multispectral camera assembly 44 further includes at least one laser 428. The multispectral camera assembly 44 includes at least one lens 430. In some embodiments, the multispectral camera assembly 44 includes two lenses 430. FIG. 15B depicts a front view of the multispectral camera assembly of FIG. 15A.

FIG. 16 depicts an exploded view of the multispectral camera assembly of FIG. 15A. As can be seen in FIG. 16, housing 410 may include a front portion 416 and a back portion 414. The front portion 416 and back portion 414 may be joined by two end caps 417.

The front portion 416 and back portion 414 may join a pitch and yaw assembly 418 situated over a base 415. The end caps 417 may be configured to smooth out the shape of the pitch and yaw assembly 418 to allow the camera assembly 44 to more easily slide through a trocar. The base 415 couples to the pitch and yaw assembly 418 and may be configured to provide a surface for rotation about the yaw axis.

The pitch and yaw assembly 418 may secure the front portion 416 while allowing articulation of the camera assembly 44 along the pitch and yaw axes. The front portion 416 may be configured to provide the pitch axis mounting features for articulation about a long axis of the housing 410.

The base 415 may be connectable to a support tube 412. The support tube 412 may connect with the motor unit of laparoscope in some embodiments, or may connect to a surgical robotic system as described above. In some embodiments, the base 415 includes a cable cover 416 configured to secure cables leading into the housing 410. For example, coaxial cables may run through the support tube 412 and extend through the base 415 and the pitch and yaw assembly 418.

The multispectral camera assembly 44 includes a camera board 432, for example a customized printed circuit board. The camera board 432 may be housed within the front portion 416. In some embodiments, the camera board 432 includes two serializer chips on a back surface of the board 432 that convert Mobile Industry Processor Interface data from one or more image sensors 433 into a serialized form that may be transmitted over two small coaxial cables. In some embodiments, the camera board 432 includes two image sensors 433 on a front surface that provide visualization of the area viewed by the camera assembly 44.

Each image sensor 433 may be aligned with a lens 430. A lens 430 may be housed within a voice coil module 434 mounted on the camera board 432. In some embodiments, the lens 430 is situated within the voice coil module 434 such that the lens 430 can be moved to change the nominal focus position of the lens 430 and to optically zoom the lens 430. The combination of image sensor 433 and lens 430 may be equivalent to camera module 124 discussed above.

The multispectral camera assembly 44 further includes a plurality of LEDs. In some embodiments, the plurality of LEDs includes at least one, for example two or four, fluorescein LEDs 426 configured to emit light to excite a fluorescent dye. Exemplary dyes may include indocyanine green, fluorescein dye, or other dyes suitable for imaging tissue or tissue structures. In some embodiments, the fluorescein LEDs 426 may emit light at a wavelength of about 490 nm, for example ranging from 475 nm to 505 nm. The fluorescein LEDs 426 may be situated near opposing ends of the camera board 432.

The plurality of LEDs further includes at least one, for example two or four, white LEDs 422 configured to emit light in the visible spectrum. The white LEDs 422 may emit light at a wavelength in a range from 400 nm to 700 nm. The white LEDSs 422 may be situated adjacent to the fluorescein LEDs 426.

The plurality of LEDs further includes at least one, for example two, blue LEDs 424 configured to emit light in a range from 400 nm to 430 nm. Each blue LED 424 may be situated between a pair of white LEDs 422. Light emitted from the blue LEDs 424 may compensate for the light blocked by notch filters, which block some of the blue spectrum, as discussed below.

The multispectral camera assembly 44 utilizes the same camera capable of white light detection for multiple spectrums by adjusting the light sourced from the LEDs 422, 424, 426. For example, the camera assembly 44 may be configured to strobe the LEDs 422, 424, 426 in sync with when a frame stops integration. This approach reduces the cost and complexity of the camera assembly 44, for example by reducing the number of necessary image sensors 433 compared to other solutions in the art.

The multispectral camera assembly 44 utilizes an image sensor's 433 entire active area for the analysis of specific bands of light. In this manner, an image is generated with the maximum white light image possible and with reasonable performance on specific bands of light. In some embodiments, the filters may be tuned to allow more infrared light. Specifically, the cutoff of the filters may be tuned to allow more or less light to enter the image sensor 433 and/or to allow more or less emitted light from the LEDs 422, 424, 426.

The multispectral camera assembly 44 may further include one or more lasers 428. Each laser may be situated near an end of the camera board 432. In some embodiments, the laser 428 is a vertical cavity surface emitting laser. The laser 428 may be configured to emit light ranging from 800 nm to 820 nm, for example 808 nm. Laser light at 808 nm enables indocyanine green (ICG) imaging by exciting indocyanine green dye. In some embodiments, the laser 428 may be configured to emit light ranging from 400 nm to 850 nm, or any range of light therebetween. In some embodiments, a filter, for example a notch filter as discussed below, may be situated in front of the laser 428.

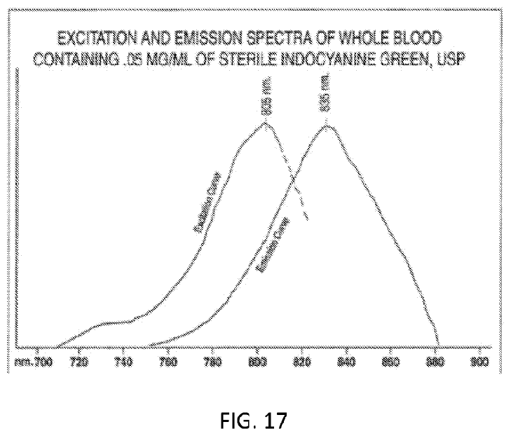

Indocyanine green dye fluoresces in the near-infrared wavelength region when illuminated by shorter-wavelength light. The dye molecules absorb excitation photons, enter an excited state, and then emit photons at longer emission wavelengths (lower energies) when the excited state collapses (as shown in FIG. 17, reproduced from “IC-GREEN™” product sheet, Akorn, Inc.). ICG has a higher probability of absorbing a photon near the peak of the excitation curve, so the wavelength of light emitted from fluorescein LEDs 426 is preferably closer to the excitation peak wavelength to increase the signal level of the emitted photons. These excitation curves can depend strongly on the environment the fluorescent compound is in. This kind of excitation curve is a typical characteristic for any fluorescent dye.

In some embodiments, the only light received by the image sensors 433 is the fluorescent emission, and not the excitation light. The fluorescence intensity may be much lower than the excitation intensity, so a notch filter 431 may be put in front of the image sensors 433 to block out the excitation light that reflects back into the camera, preventing interference.

The multispectral camera assembly 44 may include multiple notch filters 431, each notch filter 431 situated between an image sensor 433 and a lens 430. Each notch filter 431 may be configured to filter out light emitted from at least one of the plurality of LEDs. In some embodiments, the notch filter 431 filters light at or about 808 nm, 490 nm, or both. In some embodiments, a notch filter 431 may include multiple notches to filter multiple wavelengths of light. In such embodiments, a single notch filter 431 may filter light from multiple LEDs 422, 424, 426 and/or lasers 428.

The multispectral camera assembly 44 may further include bandpass filters, for example fluorescein bandpass filter 423 and laser bandpass filter 429. A fluorescein bandpass filter 423 may be situated in front of each fluorescein LED 426. In some embodiments, a fluorescein bandpass filter 423 is configured to block all light except at a wavelength around 490 nm. The laser bandpass filter 429 may be situated adjacent to the laser 428 and configured to block all light except at a wavelength around 808 nm. In some embodiments, the multispectral camera assembly 44 includes a single multi-bandpass filter positioned in front of the LEDs 422, 424, 426 and laser 428 such that specific wavelengths of light are emitted by the camera assembly 44. The LEDs 422, 424, and 426 may be interchangeable with one another.

If the camera assembly 44 is also used for visible-band (VIS) imaging, then a user may engage the laser 428 or an LED with a narrow bandpass filter in front of it (for example having a 20 nm or smaller bandwidth) so that the notch filter 431 does not have to block wavelength bands used for VIS imaging. Under these conditions, with excitation illumination on, the generated camera image is black except for the areas where fluorescence occurs. Thus the dye is used to distinguish anatomical regions that preferentially contain or absorb the dye versus those that do not.

Dyes other than ICG generally have different excitation and emission wavelengths and may be employed in order to visualize anatomies and conditions that ICG cannot. Different dyes, in general, will require different excitation and emission wavelengths. In some embodiments, the camera assembly 44 is designed to be used with multiple dyes and includes at least one filter with multiple blocking bands that do not significantly interfere with the emission bands of other dyes or the VIS camera bands. In some embodiments, the camera assembly 44 is used with multiple dyes that have the same excitation wavelength with different emission wavelengths and differentiate on an image based on the output color produced.

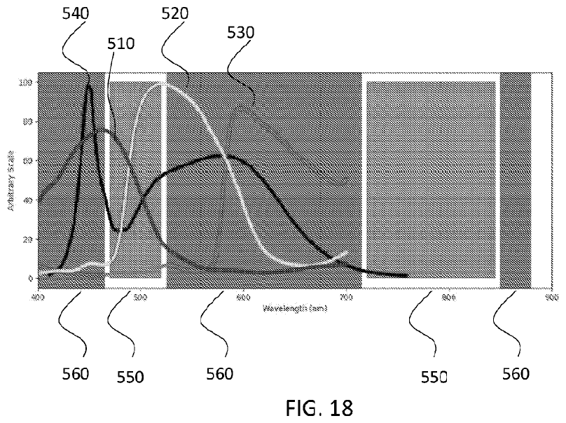

FIG. 18 is a diagram of the excitation/emission bands of ICG and fluorescein shown with the response curves of the image sensor 433's red, green, and blue channels and the emission spectrum of the white light LED 422 used for visible-band imaging. Lines 510, 520, and 530 are the pixel response curves for the image sensor 433 for the blue, green, and red channels, respectively. Line 540 is the emission characteristic of the white light LED 422 for visible band imaging. The shaded areas 550 in the diagram are the excitation bands for the dyes and blocking bands for the notch filter 431 and the clear areas 560 in the diagram are the transmission bands for the notch filter 431. The transmission bands between 400-715 nm may also be used for visible-band imaging. The indocyanine green excitation range is between 720-845 nm, and the emission range is between 850-880 nm. The fluorescein excitation range is between 470-520 nm, and the emission range is between 525-540 nm.

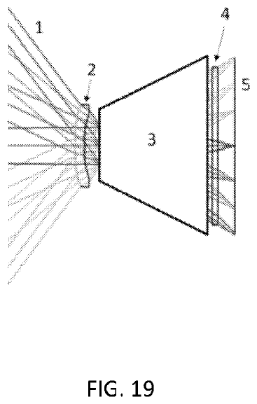

FIG. 19 illustrates light rays (1) collected over a wide angle by an illustrative camera consisting of a window (2), lens stack (3), blocking filter (4), and image sensor (5) showing the large angular extent of the light incident on the filter (4). Blocking filter characteristics change with the incident angle of the light passing into them. This change is a shift of blocking/transmission ranges to shorter wavelengths with increasing incidence angle.

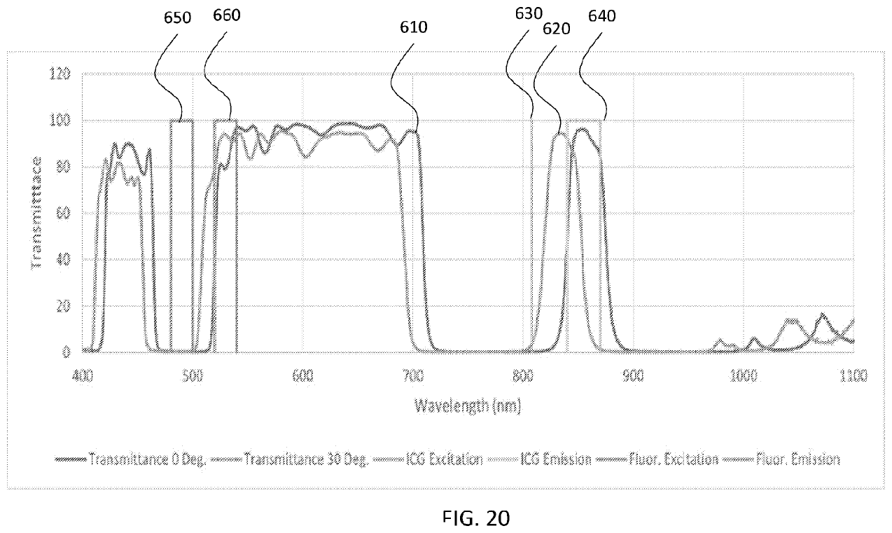

FIG. 20 depicts blocking filter characteristics 610 and 620 for incident angles of 0 and 30 degrees, respectively, showing how the fluorescein blocking range shift intrudes upon the blue emission of a white light LED. Line 630 depicts ICG excitation and line 640 depicts ICG emission. Line 650 depicts fluorescein excitation and line 660 depicts fluorescein emission. As the incident angle increases, the blocking and transmission bands shift and intrude on other bands. To compensate for this, a blue-ultraviolet LED 424 may be added to the illumination from the white LED 422 to “fill-in” the image sensor blue pixel response in the waveband 400-430 nm, where the white LED 422 has much less emission.

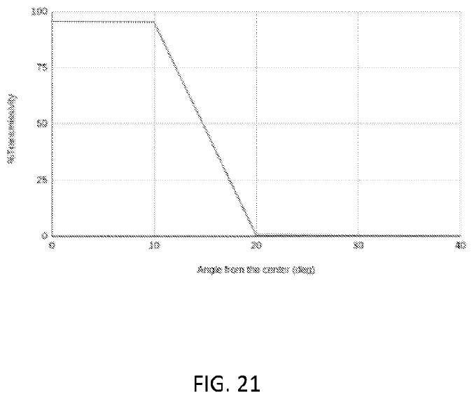

FIG. 21 depicts the dependency of transmissivity of blue light (470 nm) versus the angle from the center of a filter. As depicted, the transmissivity drops off significantly after 20 deg.

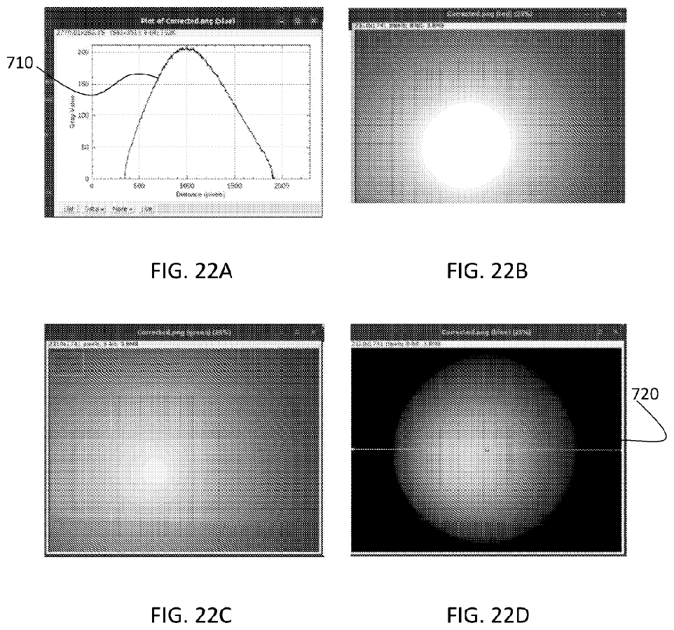

FIGS. 22A-D depict attenuation in the blue channel of an exemplary camera assembly 44 causing dark regions outside of the central 40 degree field of view. FIG. 22B depicts the red channel image of a white target illuminated by white light. FIG. 22C depicts the green channel image of the white target illuminated by white light. FIG. 22D depicts the blue channel image of the white target illuminated by white light. The attenuation in the blue channel image causes a non-uniform color in a final image of the combined channels. FIG. 22A shows the line profile 710 along the horizontal line 720 of the blue channel image of FIG. 22D. The line profile 710 shows the Gray values of the image. The Gray values are zero at about pixels 350 and 1900, resulting in the black border of the blue channel image.

Different options of illumination with increased blue intensities were explored to obtain improved transmission in the blue channel. In some embodiments, a violet LED (emitting at a wavelength of about 415 nm) is situated on or incorporated with the white LEDs 422 or blue LEDs 424. The violet LED, or any other LED suitable for illuminating a specific dye, may be included with the camera assembly 44.

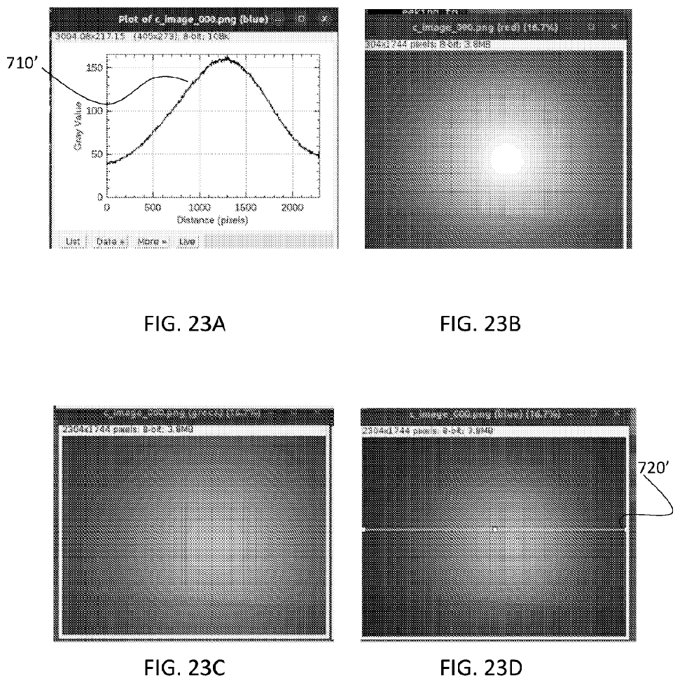

FIGS. 23B, 23C, and 23D depict channel images of a white target illuminated by white light and violet light in accordance with embodiments incorporating a violet LED. FIG. 22B depicts the red channel image of a white target illuminated by white and violet light. FIG. 22C depicts the green channel image of the white target illuminated by white and violet light. FIG. 22D depicts the blue channel image of the white target illuminated by white and violet light. FIG. 23A shows the line profile 710′ along the horizontal line 720′ of the blue channel image of FIG. 23D, showing that the cutoff of Gray values along the line profile 710′ was eliminated when employing a violet LED, eliminating the black border of the blue channel image.

The multispectral camera assembly 44 may be part of a surgical robotic system including a memory storing one or more instructions and a processor configured to or programmed to read the one or more instructions stored in the memory. The processor may be operationally coupled to the one or more camera assemblies 44 to capture multiple spectrums of light simultaneously from the camera assemblies 44. The system may be operably connected to a display to depict the images captured by the system.

Digital cameras known in the art are designed to integrate a frame for a set period of time and read that frame into memory and transmit that frame to a display. One of the issues in surgical applications is the amount of data and time it takes to process or transmit each frame. In some instances up to 128 megabytes of information are required to transmit and process per frame. Global shutter cameras are able to store that information all at once, transfer that information and then process a new frame while transmitting. Therefore global shutter cameras require more energy, storage, and complicated electronics than other solutions. Sensors associated with global shutter cameras usually have lower resolution, lower frames per second, and higher power draw. Such sensors are not typically used on mobile devices due to these tradeoffs. But such sensors would allow for trivial timing of strobing lights to change between integration sections.

In some embodiments, the camera assembly 44 incorporates global shutter cameras, for example a global shutter charge-coupled device (CCD) imaging sensor. In some embodiments, the camera assembly 44 incorporates rolling shutter cameras as opposed to global shutter cameras, for example a rolling shutter complementary metal oxide semiconductor (CMOS) imaging sensor.

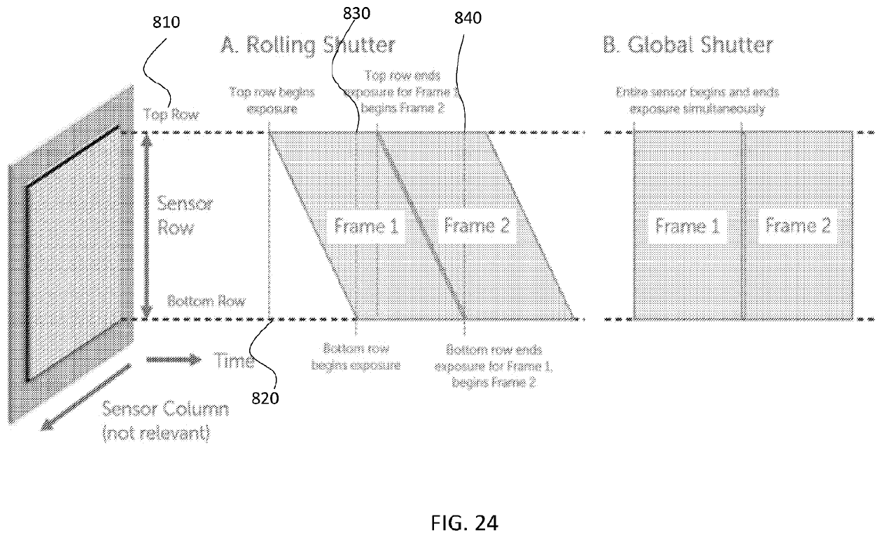

FIG. 24 depicts the integration time and operation of frame one and frame two of illustrative rolling camera shutters. The graphic depicts the timing of the top row 810 and bottom row 820 integration. As shown in FIG. 24, if the frame between transitions is dropped (or adjusted such that exposure is minimized) the controller 26 need only be configured to trigger a transition between images anytime between the N-2 frame, which stops the bottom row 820 of integration and the N frame, which begins integration of the top row 810. The transition time as described above needs to be after the depicted first vertical dotted line 830 and before the depicted second vertical dotted line 840.

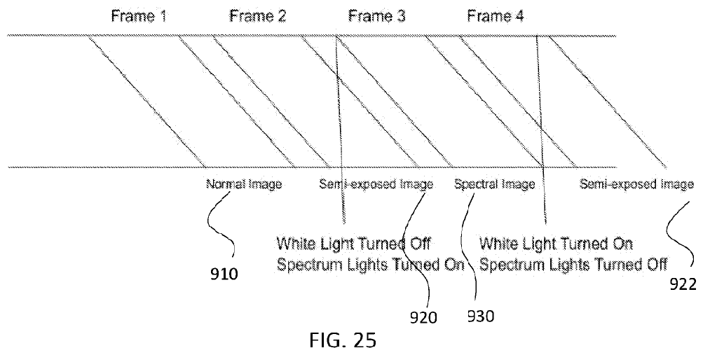

When combined with strobing of one or more LEDS, a rolling shutter enables processing of multiple spectrums of light simultaneously by the camera assembly 44. FIG. 25 provides an example of transition frames combined with normal, semi-exposed, and spectral images.

FIG. 25 depicts a pattern of a normal image 910, a first semi-exposed image 920, a spectral image 930, and a second semi-exposed image 922 in sequence that may be employed by the camera assembly 44 in embodiments incorporating rolling shutters. The system 100 may be configured to transition from one spectral image 930 to another spectral image 930 or change the number of spectral images 930 or normal images 910 to provide any desired ratio.

In some embodiments, the camera assembly 44 may provide a multispectral video on a display 130 or headset 140. Creation of the video may involve three components: the camera assembly 44, the controller 26 that controls the camera assembly 44, and a graphics processing unit (GPU) 52 configured to process video data, user input, and camera information to control the system 100 at a high level and display video output to the user. Functionality performed by the GPU 52 may be performed by the graphics processing unit specifically and/or by a vision processing unit containing the graphics processing unit.

The image sensors 433 may be initialized into a state that provides images in the correct format for producing a video. This state must also provide metadata of each frame that allows the controller 26 and the GPU 52 to coordinate timing of frames. Other than those two constraints, the image sensors 433 can be thought of as a data producer for the purposes of understanding the software processing.

The controller 26 may be configured to initialize the electronics of the camera assembly 44, as well as passing commands from the GPU 52 to the camera assembly 44. The controller 26 may be configured to listen for timing information from the image sensors 433, and depending on the current visualization mode, may use that information to drive multispectral imaging modes. There may not be tight coupling between the software on the controller 26 and the GPU 52; instead the GPU 52 may send lighting and image sensor 433 commands when requested, and may use the metadata coming out of the sensors 433 to understand the state of the system 100.

The GPU 52 may be configured to control all image processing, user input, and display output. The GPU 52 may consume image data and corrects initial issues, converting the image data to a format that can be processed more easily. The controller 26 may then send the image data into a modular parallel-processing based video pipeline, which performs rectification, color correction, overlay processing, and then presents that processed image data to the user on a display 130 or headset 140. The GPU 52 also may be configured to take any information from the processed image and use that information to send commands to the controller 26, such as alterations in focus or lighting levels.

The controller 26 controls the point of light source change over by analyzing the timing provided by the image sensor's 433 frame start packet and frame end packet along with the frame sync signal. The start and end packets are intercepted as transmitted by the sensor 433 over csi-2 to provide more precise information on the frame blanking section where the controller 26 may be configured to swap light sources to use. The precise time used is important to ensure that each image doesn't have bleeding from the other main image. Timing is even more important when timing the activation of the interlaced frame for use in high-dynamic range imaging.

Image processing modules of the controller 26 and GPU 52 can be removed or added depending on the needs of the user in producing video output. In some embodiments, modules may include rectification, color correction, depth detection, luminance detection, overlay processing, and compositing for video output. In the depth and luminance detection stages of image processing, the outputs of those modules may be used by a GPU 52 to send commands to the controller 26 to control focus and lighting respectively.

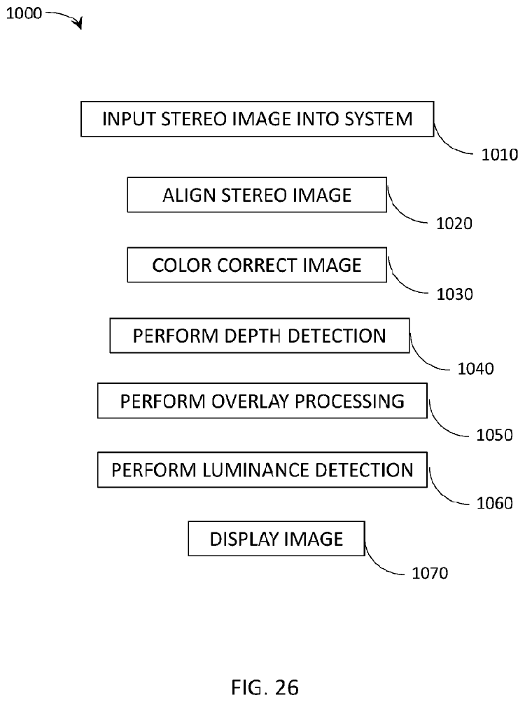

A flowchart of a method 1000 of image processing to display a multispectral image is depicted in FIG. 26. The method 1000 may begin at Step 1010 when an image, for example from the image sensor 433, is input into the system 100. The image may be a stereo image, a pair of images taken next to each other such that they could be a left eye and a right eye. At Step 1020, a stereo image is aligned so that the image is comfortable for a human to view. Alignment ensures that any features on one line of a left eye image are going to be on the same vertical pixel line of the right eye image. Stereoscopic cameras have manufacturing defects, misalignment, other issues that make it uncomfortable to view them through a VR headset, 3D monitor, etc. This process is well-documented and the system 100 may use a standard algorithm to perform it.

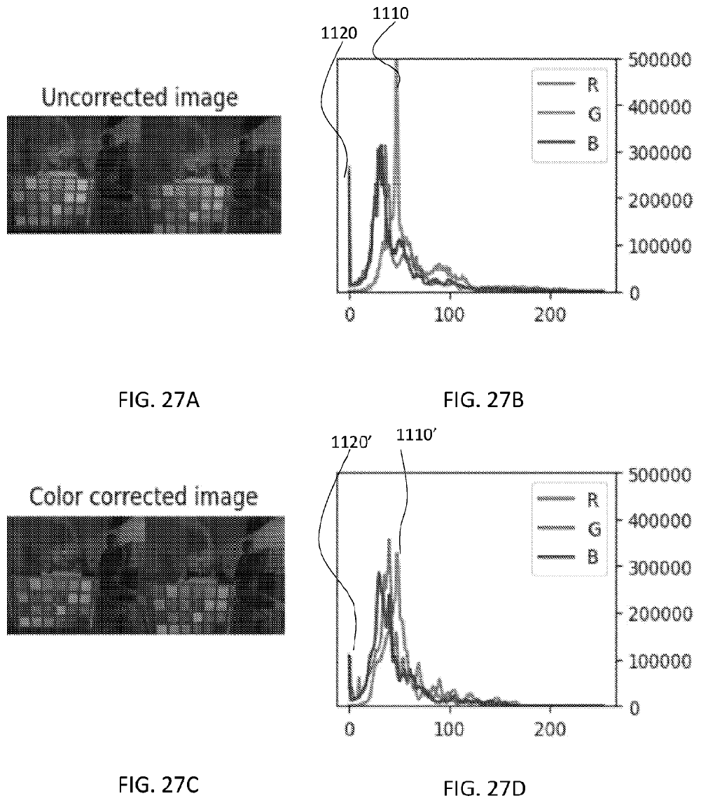

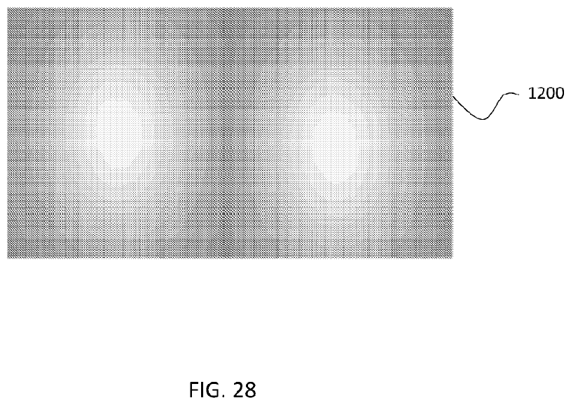

At Step 1030, the image may be color corrected. Color correction is the use of multiple colors of light illuminating the object and color selective filters attached to the image sensors 433. Exemplary color correction is depicted in FIGS. 27A-27D. FIG. 27A shows an image of a color calibration chart before color correction. The histogram of FIG. 27B shows two peaks, a red channel peak 1110 near the intensity value of fifty and a blue channel peak 1120 at the intensity value of zero. A non-uniformity correction, as shown in FIG. 27C, which shows an image with a more muted red channel, may be carried out by dividing the image by a flat field image acquired with a uniformly illuminated field. The histogram of FIG. 27D depicts the peaks 1110′, 1120′ associated with the correction. An exemplary flat field image 1200 is depicted in FIG. 28. The color correction matrix may be generated using, for example, an Imatest® color correction module and may be applied to the image after non uniformity correction.

At Step 1040 the system 100 may be perform depth detection. Depth detection is the process of taking a stereo image and determining the distance to each point in the image. The system 100 may employ a machine learning model to determine the distance at each pixel, and then use that information to choose a focus distance. In calibration, the system 100 determine how to set focus for any given distance, for example using the distance of the center of the screen to automatically focus where the surgeon is looking. In some embodiments, a depth perception node of the GPU 52 also may send a callback out to a messaging system when Step 1040 finishes, transmitting the appropriate focus distance over the network of the system 100.

Overlay processing may be performed at Step 1050. In some embodiments, overlay processing may be performed by the overlay processing module of the GPU 52. The overlay processing module may have different functions depending on the input. Fluorescent frames may be converted into an overlay and stored in a temporary buffer. Color frames may have the current overlay buffers added to them.

Fluorescent frames may be turned into a transparent overlay. Each fluorescent dye produces a response on an image sensor 433 that can be uniquely identified. Dependent upon the type of fluorescent light used for each frame, the frame can be processed into a mask, where areas with fluorescent dye are marked with white, and areas without fluorescent dye are dark. These mask images may be used to add extra information to the color frames. The system 100 may store them in a temporary location, one per type of fluorescent frame in use by the system 100.

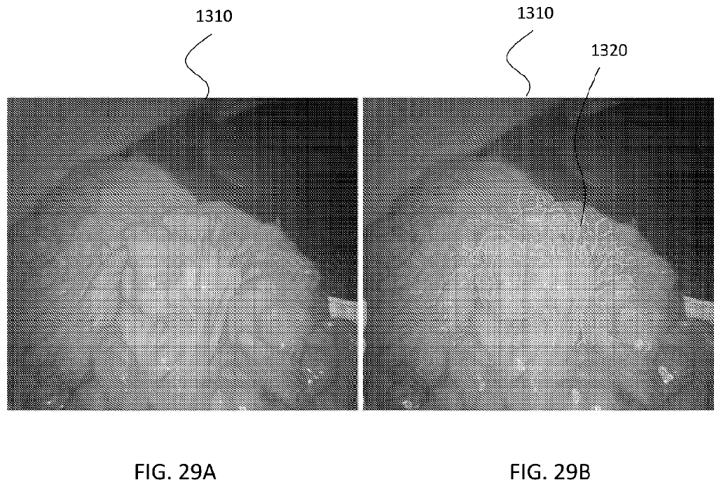

Color frames may not be processed directly, but instead may have the mask images overlaid on top of them. The mask images may be black and white images, which may be colored depending on a color key for fluorescent frames. FIG. 29A depicts a color frame 1310 with no overlay and FIG. 29B depicts a color frame 1310 having a fluorescein mask image 1320 overlaid on top of it. In some embodiments, the fluorescein mask image 1320 may be colored green to stand out against the background.

The overlaid frames may receive the latest processed mask from all current types of fluorescent imaging. In exemplary embodiments using ICG and fluorescein, both masks may be overlaid on top of the original image. The overlays enable a user to pick out information on the image (for example blood vessels, ureters, or other anatomical structures) that otherwise the surgeon may have difficulty identifying. In other words, the overlays provide a relief of the surgeon's cognitive load.

At Step 1060, the system may be configured to perform luminance detection to calculate how bright an image appears on screen. By quantifying how bright the image is, a surgeon can adjust the amount of light emitted from the LEDs to a brightness specified by the surgeon. The system 100 may be configured to receive user input for how bright the image should be lit, and then the system 100 may employ luminance detection to inform a control loop on the GPU 52. The GPU 52 may send a callback out to the messaging system when the luminance detection calculation is finished to transmit the calculation to the surgeon.

At Step 1070, an image composition module of the GPU 52 may employ a framework for displaying the image on the display 130 or headset 140 called OpenGL. OpenGL allows for basic operations like drawing images to a screen, and has built in methods for arranging different images (called textures) on a larger screen. The system 100 may utilize OpenGL to arrange the left and right eyes on the display 130 or on the headset 140.

Steps 1020, 1030, 1040, 1050, 1060 may be performed in a different order than the order presented in FIG. 26.

Between each normal frame and a spectral frame, an interlaced frame may be generated as the illumination is switching between spectral and white LEDs. These frames contain a mix of normal and spectral data. These frames have useful information which can be incorporated into the images being presented to the user.

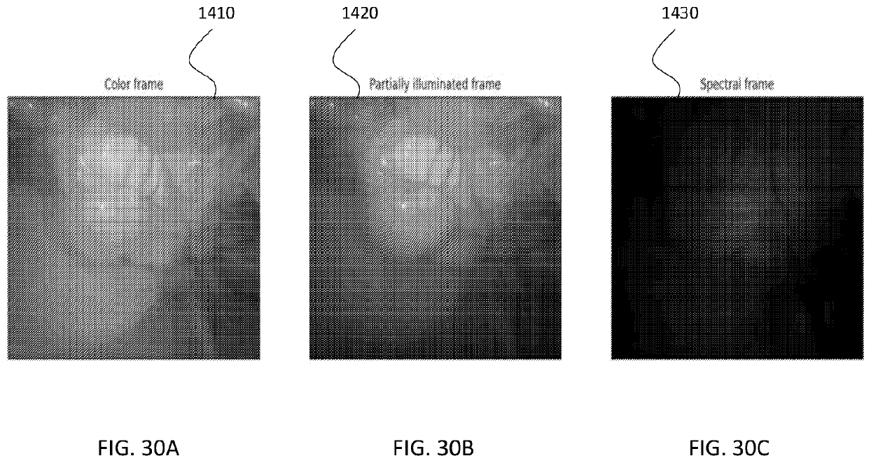

FIGS. 30A-30C depict the interlaced frame 1420 between a normal frame 1410 and a spectral frame 1430. The interlaced frame 1420 is partially illuminated resulting in a darker and noisier image.

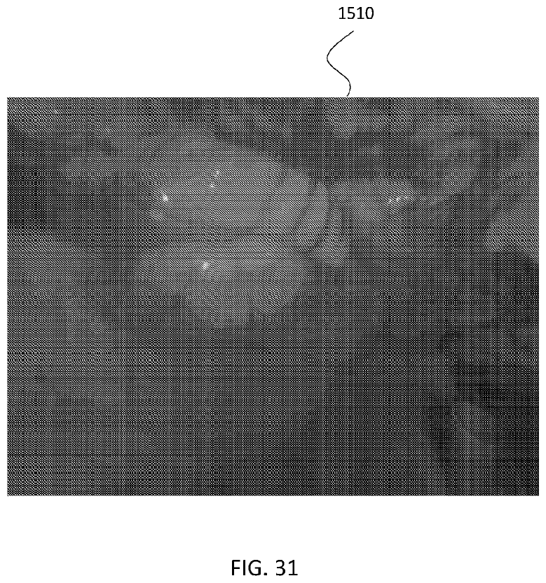

In a sequential acquisition, the normal frame 1410 may also preceded by another partially illuminated interlaced frame 1420. Both the interlaced frames 1420 may have different illumination as the illumination sequence is different. As a result, both interlaced frames 1420 have different normal and noise characteristics preventing the system 100 from using conventional methods to combine the frames. The system may adapt the Debevec algorithm of creating HDR images. The algorithm as published may be applied to conventional color and grayscale images. Conventional HDR images may be produced by using images acquired by the same image sensor 433 with different exposures under the same lighting conditions. As the interlaced frames 1420 are illuminated by different color spectra, the system 100 may adapt the algorithm to introduce a different scheme of applying weights to RGB channels as well as modulating the global exposure weights. These weights can be obtained by observing the color vectors in the interlaced images. Each of the interlaced frames 1420 will have a different color correction matrix allowing the system 100 to derive appropriate weights to combine the frames. FIG. 31 depicts the result of combining two interlaced and one normal frame between two spectral frames of the same light source. More specifically, FIG. 31 depicts a tone mapped combined image 1510 of two partially illuminated frames and one normal frame. The advantage of combining images in this manner is that in addition to utilizing interlaced frames, the system 100 is able to recover significant amounts of information which gets washed out in normal frames.

The interlaced frames 1420 are illuminated with white light as well as the wavelength that induces fluorescence in the injected dye. Different dyes have different fluorescence wavelengths. For example, fluorescein emission is at peak of 525 nm which appears green and ICG emission is at peak of 814 nm which appears purple after going through the filters. Depending on the dye selection, theoretically it is possible to separate the colors from the partially exposed interlaced frame in the frequency domain. These color vectors will appear as peaks among other frequencies which will be more uniformly distributed. With frequency selective digital filtering techniques it is possible to extract the emission data from the interlaced images 1420. This data can then be combined with the spectral image 1430 to reduce the noise in the spectral image 1430.

While preferred embodiments of the present invention have been shown and described herein, it will be obvious to those skilled in the art that such embodiments are provided by way of example only. Numerous variations, changes, and substitutions will now occur to those skilled in the art without departing from the invention. It may be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A camera assembly configured for simultaneous multispectral imaging, comprising:
a first lens assembly;
a second lens assembly;
a first plurality of light emitting diodes (LEDs) configured to emit light in a first wavelength range;
a second plurality of LEDs configured to emit light in a second wavelength range;
a plurality of LED bandpass filters, a respective one of the plurality of LED bandpass filters situated in front of each of the second plurality of LEDs to filter light emitted therefrom;
a plurality of image sensors, a first of the plurality of image sensors positioned behind the first lens assembly to capture light therefrom, and a second of the plurality of image sensors positioned behind the second lens assembly to capture light therefrom;
a plurality of notch filters, each notch filter situated between a respective one of the plurality of image sensors and either the first lens assembly and the second lens assembly, each notch filter configured to filter out light in a selected wavelength range transmitted by the respective first and second lens assembly; and
a circuit board electronically coupled to the first and second plurality of LEDs, and the plurality of image sensors, the circuit board configured to strobe the plurality of LEDs such that each of the plurality of image sensors captures multiple spectrums of light simultaneously.

2. The camera assembly of claim 1, further comprising a laser.

3. The camera assembly of claim 2, further comprising a laser bandpass filter situated adjacent to the laser to allow a selected wavelength band of light form the laser pass therethrough.

4. The camera assembly of claim 1, wherein the first plurality of LEDs is configured to emit light in a range from 400 nm to 700 nm and the second plurality of LEDs is configured to emit light in a range from 800 nm to 820 nm.

5. The camera assembly of claim 1, further comprising a third plurality of LEDs configured to emit light in a range from 475 nm to 505 nm.

6. The camera assembly of claim 1, wherein at least one of the plurality of LED bandpass filters is configured to block all light except at a wavelength around 490 nm.

7. The camera assembly of claim 1, wherein the second plurality of LEDs is configured to excite a dye in biological tissue.

8. The camera assembly of claim 7, wherein the dye is fluorescein dye.

9. The camera assembly of claim 1, wherein at least one of the plurality of LED bandpass filters is configured to allow passage of visible light.

10. A surgical robotic system, comprising:
a first camera assembly having one or more LEDs, one or more lens, one or more filter elements and one or more imaging sensors; and
a second camera assembly having one or more LEDs, one or more lens, one or more filter elements and one or more imaging sensors, the first and second camera assembly providing stereoscopic images for viewing by a user of the system;
a memory storing one or more instructions; and
a processor configured to or programmed to read the one or more instructions stored in the memory, the processor operationally coupled to the first camera assembly and the second camera assembly to capture multiple spectrums of light simultaneously from the first and the second camera assembly.

11. The surgical robotic system of claim 10, further comprising a display operably connected to the first camera assembly and the second camera assembly, the display configured to depict an image captured by the one or more imaging sensors of each camera assembly.

12. The surgical robotic system of claim 11, wherein the processor is configured to strobe the plurality of LEDs such that the image is made up of multiple spectrums of light.

13. The surgical robotic system of claim 10, wherein at least one of the first camera assembly or the second camera assembly further comprises a laser.

14. The surgical robotic system of claim 13, wherein at least one of the first camera assembly or the second camera assembly further comprises a laser bandpass filter situated adjacent to the laser to allow a selected wavelength band of light from the laser pass therethrough.

15. The surgical robotic system of claim 10, wherein the one or more LEDs of at least one of the first camera assembly or the second camera assembly includes at least one LED configured to emit light in a range from 400 nm to 700 nm and at least one LED configured to emit light in a range from 800 nm to 820 nm.

16. The surgical robotic system of claim 15, wherein the one or more LEDs of at least one of the first camera assembly or the second camera assembly further includes at least one LED configured to emit light in a range from 475 nm to 505 nm.

17. The surgical robotic system of claim 10, wherein the one or more filter elements of at least one of the first camera assembly or the second camera assembly are configured to block all light except at a wavelength around 490 nm.

18. The surgical robotic system of claim 10, wherein the one or more LEDs of at least one of the first camera assembly or the second camera assembly is configured to excite a dye in biological tissue.

19. The surgical robotic system of claim 18, wherein the dye is fluorescein dye.

20. The surgical robotic system of claim 10, wherein the one or more filter elements of at least one of the first camera assembly or the second camera assembly are configured to allow passage of visible light.
